(19) United States
(12) Patent Application Publication
(10) Pub. No.: US 2006/0110468 A1
(43) Pub. Date: May 25, 2006

(54) Herbal formulations for arthritis

(76) Inventors: Liang Liu, Hong Kong (HK); Hua Zhou, Hong Kong (HK); Yuen Fan Wong, Hong Kong (HK)

Correspondence Address: KNOBBE MARTENS OLSON & BEAR LLP, 2040 MAIN STREET, FOURTEENTH FLOOR, IRVINE, CA, 92614, US

(21) Appl. No.: 10/997,801

(22) Filed: Nov. 24, 2004

(51) Int. Cl. A61K 36/9066; A61K 36/45

(52) U.S. Cl. 424/725; 424/756; 424/773

(57) ABSTRACT

The present invention provides an oral formulation comprising ingredients extracted from Caulis Sinomenii, Radix Aconiti Lateralis Preparata, Radix Paeoniae Alba, Cortex Moutan, and Rhizoma Curcumae Longae. The formulation has anti-arthritic, anti-inflammatory and anti-nociceptive properties and is suitable for the treatment of arthritis, symptoms associated with arthritis and other similar conditions.

### FIELD OF INVENTION

The present invention is related in general to the field of dietary supplements and therapeutic compounds. In particular, the present invention relates to formulations comprising herbal extracts for the amelioration of arthritis and for the alleviation of inflammation and pain associated with arthritis.

### BACKGROUND OF INVENTION

References which are cited in the present disclosure are not necessarily prior art and therefore their citation does not constitute an admission that such references are prior art in any jurisdiction. All publications, patents and patent applications herein are incorporated by reference to the same extent as if each individual or patent application was specifically and individually indicated to be incorporated by reference.

Arthritis is a disease affecting the musculoskeletal system, particularly the joints. It is not a single disease but actually an umbrella term for over 100 medical conditions where the joints are affected. According to the Arthritis Foundation, arthritis afflicts more than 70 million adults and 300,000 children in the United States alone.

One form of arthritis is rheumatoid arthritis (RA). RA is a systemic disease that affects the entire body and is one of the most common forms of arthritis. RA is a chronic inflammatory and destructive joint disease that affects 0.5˜1.0% of the population in the industrialized world and commonly leads to significant disability and a consequent reduction in quality of life. It is two to three times more frequent in women than in men and can start at any age, with a peak incidence between the fourth and sixth decades of life. RA is associated with high costs and, if not treated appropriately, with a reduction in life expectancy. In addition to the joint swelling and pain caused by the inflammatory process, extra-articular involvement, which ranges from rheumatoid nodules to life threatening vasculitis, is a characteristic of RA. The ultimate hallmark of RA is joint destruction.

Another form of arthritis is ankylosing spondylitis (AS). AS is a rheumatic disease that causes arthritis of the spine and sacroiliac joints and can cause inflammation of the eyes, lungs, and heart valves. It varies from intermittent episodes of back pain that occur throughout life to a severe chronic disease that attacks the spine, peripheral joints and other body organs, resulting in severe joint and back stiffness, loss of motion and deformity as life progresses. AS afflicts an estimated 129 out of 100,000 people in the United States and typically strikes adolescents and young adult males. The prevalence of AS varies by ethnic group and is most common in Native Americans.

Several patents have been issued for topical medicaments to relieve the pain and inflammation caused by arthritis and other similar diseases. For example, U.S. Pat. No. 6,274,176 (Tomer) teaches an edible formulation comprising Tanacetum parthenium, Zingibar officinale, Curcuma longa, Coriander sativum, Centella asiatica, Oenothera biennis, Valeriana officinalis, Tabebuia impetiginosa, Thymus vulgaris and Sambucus nigra.

Another patent, U.S. Pat. No. 6,350,476 granted to Hou, teaches a formulation comprising ingredients from the plant species of the genera Stephania, Coix, Pinellia, Prunus, Phellodendron, Sophora, Tetrapanax, Stemona, Glycyrrhiza, Tripterygium, Forsythia, and Siegesbeckia. Interestingly, an earlier patent granted to Hou (U.S. Pat. No. 5,908,628) teaches the same formula as the later patent but has the addition of talc and silkworm excrement. The invention subsequently found that the talc and silkworm excrement were not essential for the invention to work. That discovery formed the basis of the later invention protected by U.S. Pat. No. 6,350,476.

Other inventions for similar application use many herbal ingredients. Chinese Patent No 98125678.3 by Ren uses a formulation of 27 ingredients, namely Herba Dendrobii, Radix Rehmanniae Preparata, Radix Aconiti Lateralis Preparata, Radix Dipsaci, Fructus Psoraleae, Radix Tripterygii Wilfordii, Caulis Sinomenii, Ramulus Cinnamomi, Rhizoma Notopterygii, Radix Angelicae Pubescentis, Radix Paeoniae Alba, Radix Clematidis, Herba Lycopodii, Radix Astragali seu Hedysari, Radix Stephaniae Tetrandrae, Radix Saposhnikoviae, Rhizoma Atractylodis, Rhizoma Atractylodis Macrocephalae, Herba Asari, Lumbricus, Caulis Spatholobi, Radix Paeoniae Rubra, Rhizoma Anemarrhenae, Eupolyphaga seu Steleophaga, Radix Linderae, Radix Glycyrrhizae, and Radix Achyranthis Bidentatae in an ointment that reportedly is effective in reducing pain and inflammation.

Another invention, Chinese Patent No 99120466.2 by Wang is an externally applied medicament comprising 43 traditional Chinese medicinal products Rhizoma Gastrodiae, Radix Notoginseng, Cortex Eucommiae, Herba Epimedii, Eupolyphaga seu Steleophaga, Radix Aconiti, Radix Aconiti Kusnezoffii, Scorpio, Scolopendra, Radix Polygoni Multiflori, Squama Manitis, Hirudo, Olibanum, Myrrha, Radix Angelicae Sinensis, Rhizoma Chuanxiong, Radix Salviae Miltiorrhizae, Semen Persicae, Radix Paeoniae Rubra, Caulis Spatholobi, Flos Carthami, Resina Draconis, Radix Dipsaci, Herba Taxilli, Cortex Moutan, Rhizoma Curcumae Longae, Radix Cyathulae, Cortex Acanthopanax Radicis Zaocys, Corium Elaphatis Crinis Carbonisatus, Herba Ephedrae, Herba Asari, Radix Saposhnikoviae, Radix Angelicae Pubescentis, Radix Angelicae Dahuricae, Rhizoma Pinelliae seu Arisaematis, Radix et Rhizoma Rhei, Radix Puerariae, Pericarpium Papaveris, Borneolum, Semen Brassicae Junceae, Lead Sesquioxide and Oleum Sesami to achieve similar effects.

While such externally-applied medicaments may have some effects on the treatment of arthritis, the effects are only seen insofar as the ability of the medicament to penetrate the superficial tissue into the muscles and joints when applied externally. It is therefore an object of the present invention to further improve a herbal medicament for arthritis.

### SUMMARY OF INVENTION

This present invention provides, in one aspect, an oral formulation for the alleviation of the symptoms associated with arthritis or an oral formulation for the prophylactic amelioration of arthritis. The present formulation also provides methods of extracting useful compounds from herbs for use in these oral formulations.

In some embodiments of the present invention, the formulation comprises extracts of: Sinomenium spp., Aconitum carmichaeli Debx, Paeoniae lactiflora Pall, Paeonia suffruticosa Andr. and Curcuma longa L. Specifically, the formulation comprises extracts of Caulis Sinomenii, Radix Aconitii Lateralis Preparata, Radix Paeoniae Alba, Cortex Moutan and Rhizoma Curcumae Longae.

In some embodiments of the present invention, the formulation comprises essentially of Caulis Sinomenii, Radix Aconitii Lateralis Preparata, Radix Paeoniae Alba, Cortex Moutan and Rhizoma Curcumae Longae.

In some embodiments of the present invention, the formulation comprises Caulis Sinomenii, Radix Aconitii Lateralis Preparata, Radix Paeoniae Alba, Cortex Moutan and Rhizoma Curcumae Longae.

In a preferred embodiment, the formulation comprises plant materials in the following proportions by weight as follows: Caulis Sinomenii: about 2-10 parts, Radix Aconitii Lateralis Preparata: about 1-6 parts, Radix Paeoniae Alba: about 3-15 parts, Cortex Moutan: about 1-8 parts and Rhizoma Curcumae Longae: about 1-8 parts, or the equivalent amounts of fresh plant materials of these respective plants in the proportion taught.

In one implementation of the invention, the formulation of the invention comprises plant materials in the following proportion by weight of about: Caulis Sinomenii: about 5 parts, Radix Aconiti Lateralis Preparata: about 3 parts, Radix Paeoniae Alba: about 6 parts, Cortex Moutan: about 3 parts, and Rizhoma Curcumae Longae: about 3 parts.

In another implementation, the formulation of the invention comprises plant materials in the following proportion by weight of about: Caulis Sinomenii: about 4 parts, Radix Aconiti Lateralis Preparata: about 3 parts, Radix Paeoniae Alba: about 5 parts, Cortex Moutan: about 3 parts, and Rhizoma Curcumae Longae: about 2 parts.

In another aspect, the invention describes a method of reducing the size of the herbs Caulis Sinomenii, Radix Aconitii Lateralis Preparata, Radix Paeoniae Alba, Cortex Moutan and Rhizoma Curcumae Longae, extracting the herbs with suitable solvents, and concentrating the herbs to form a mixture for use as an oral formulation.

In a first preferred embodiment of this aspect of the present invention, the reducing step is preferably done by pulverizing the herbs. The extraction step comprises refluxing Caulis Sinomenii, Radix Aconitii Lateralis Preparata and Radix Paeoniae Alba together with an alcohol to produce an Extract 1; extracting Cortex Moutan with supercritical carbon dioxide to produce an Extract 2; extracting the residue of Cortex Moutan with an alcohol to form an Extract 3; extracting Rhizoma Curcumae Longae with supercritical carbon dioxide to produce an Extract 4; and extracting the residue of Rhizoma Curcumae Longae with an alcohol to form an Extract 5. These extracts can then be used individually or in combination. In a preferred embodiment, a combined extract is formed by combining Extracts 1, 2, 3, 4 and 5.

In the teachings of the present invention, any of these extraction methods can be followed by a purification process, preferably by passage through a polymeric adsorption resin, to obtain a purified extract.

Another preferred embodiment of the invention involves the steps of reducing in size Caulis Sinomenii, Radix Aconiti Lateralis Preparata, Radix Paeoniae Alba, Cortex Moutan, and Rizhoma Curcumae Longae; obtaining an aqueous extract of Caulis Sinomenii, obtaining an alcoholic extract of Radix Aconiti Lateralis Preparata and Radix Paeoniae Alba, obtaining a supercritical fluid extract of Cortex Moutan, obtaining an alcoholic extract of Cortex Moutan, obtaining a supercritical fluid extract of Rizhoma Curcumae Longae, obtaining an alcoholic extract of Rizhoma Curcumae Longae as an Extract 6; and mixing these extracts with suitable excipients to obtain a formulation suitable for filling capsules for oral administration.

In yet another aspect, the present invention provides a composition comprising several main active ingredients extracted from Caulis Sinomenii, Radix Aconitii Lateralis Preparata, Radix Paeoniae Alba, Cortex Moutan and Rhizoma Curcumae Longae; these several main active ingredients being sinomenine, paeoniflorin, peonol, and curcumin. The composition can further comprise demethoxycurcumin and bisdemethoxycurcumin.

The present invention also provides, in another aspect, a method of treating arthritis in a mammal comprising orally administering a therapeutically effective amount of the composition given above or obtained by the method taught above.

The present invention also teaches a composition described herein for use in a medicament for the treatment of arthritis in a mammal. Thus, the present invention also relates in a further aspect to a medicament or health supplement that can be orally and systemically applied to the entire body to exert its effects in deeper tissues in the treatment of arthritis and arthritic conditions such as those due to rheumatoid arthritis and ankylosing spondylitis. The present invention also provides method and criteria of controlling the quality of the products of the present invention through chromatographic analyses.

In some embodiments of the invention, a formulation having the extracts of Sinomenium spp., Aconitum carmichaeli Debx, Paeonia lactiflora Pall, Paeonia suffruticosa Andr. and Curcuma longa L. is provided. The Sinomenium spp. can be selected, for example, from a group consisting of Sinomenii acutum (Thumb.) Rehd. et Wils. and Sinomenii acutum (Thumb.) Rehd. et Wils. var. cinereum Rehd. et Wils. The formulation can contain the extracts of Caulis Sinomenii, Radix Aconitii Lateralis Preparata, Radix Paeoniae Alba, Cortex Moutan and Rhizoma Curcumae Longae. In some embodiments, the plant materials can be extracted the proportion by weight of about: Caulis Sinomenii 2-10 parts, Radix Aconitii Lateralis Preparata 1-6 parts, Radix Paeoniae Alba 3-15 parts, Cortex Moutan 1-8 parts and Rhizoma Curcumae Longae 1-8 parts. In some embodiments, the plant materials can be extracted in the proportion by weight of about Caulis Sinomenii 5 parts, Radix Aconiti Lateralis Preparata 3 parts, Radix Paeoniae Alba 6 parts, Cortex Moutan 3 parts, and Rizhoma Curcumae Longae 3 parts. In additional embodiments, the plant materials can be extracted in the proportion by weight of about Caulis Sinomenii 4 parts, Radix Aconiti Lateralis Preparata 3 parts, Radix Paeoniae Alba 5 parts, Cortex Moutan 3 parts, and Rizhoma Curcumae Longae 2 parts. The formulation can additionally have a pharmaceutically-acceptable carrier, diluent or additive, and may be in a form that is suitable for oral administration. The formulation may be in the form of, for example, a capsule, powder, tablet, liquid or caplet. In some embodiments, the formulation can also have therapeutically effective amounts of sinomenine, paeoniflorin, peonol and curcumin in the following proportions by weight: sinomenine 1-5 parts, paeoniflorin 5-28 parts, peonol 1-10 parts and curcumin 1-8 parts. The formulation can also have therapeutically effective amounts of demethoxycurcumin and bisdemethoxycurcumin. The formulation can be, for example, a nutraceutical. Methods of ascertaining the quality of the formulation are also provided, by providing an extract of the formulation, subjecting the extract to at least one separation technique under certain parameters, subjecting at least one known marker to the same at least one separation technique under the same certain parameters, where the quality of the formulation may be ascertained by comparing the at least one known marker with a corresponding marker in the extract.

Additional embodiments of the invention provide a method of preparing a formulation, the method having the steps of reducing the size of Caulis Sinomenii, Radix Aconitii Lateralis Preparata, Radix Paeoniae Alba, Cortex Moutan and Rhizoma Curcumae Longae; extracting Caulis Sincomenii, Radix Aconitii Lateralis Preparata, Radix Paeoniae Alba, Cortex Moutan and Rhizoma Curcumae Longae with suitable liquids to form extracts; concentrating the extracts; and combining the above extracts. The extraction step can also include the following steps: extracting Caulis Sinomenii, Radix Aconitii Lateralis Preparata, Radix Paeoniae Alba, together with an alcohol to produce an Extract 1; extracting Cortex Moutan with supercritical carbon dioxide to produce an Extract 2; and extracting Rhizoma Curcumae Longae with supercritical carbon dioxide to produce an Extract 3. The extracting Cortex Moutan step can be followed, for example, by extracting the Cortex Moutan residue with an alcohol to produce another extract. The extracting Rhizoma Curcumae Longae step can be followed, for example, by extracting the Rhizoma Curcumae Longae residue with an alcohol to produce another extract. In some embodiments, the method involves reducing in size Caulis Sinomenii 62.5 g, Radix Aconiti Lateralis Preparata 37.5 g and Radix Paeoniae Alba 75 g, Cortex Moutan 37.5 g and Rizhoma Curcumae Longae 37.5 g; refluxing the Caulis Sinomenii, Radix Aconiti Lateralis Preparata and Radix Paeoniae Alba with 1250 ml of 80% ethanol 3 times for 1 hour for each time to obtain ethanolic extracts; combining the ethanolic extracts; filtering the ethanolic extracts; concentrating the filtered ethanolic extracts by vacuum evaporation to a concentration of 100% (W/v) to produce 175 ml of an Extract 1; extracting Cortex Moutan with supercritical carbon dioxide to produce an Extract 2; refluxing the Cortex Moutan residue with 187.5 ml of 80% ethanol 3 times for 1 hour each time to obtain ethanolic Cortex Moutan extracts; combining and filtering the ethanolic Cortex Moutan extracts; concentrating the filtered ethanolic Cortex Moutan extracts by vacuum evaporation to a concentration of 100% (w/v) to produce 37.5 ml of an Extract 2a; extracting the Rizhoma Curcumae Longae extracted by supercritical carbon dioxide to produce 2.8 ml of an Extract 3; refluxing the Rizhoma Curcumae Longae residue with 187.5 ml of 80% ethanol 3 times for 1 hour for each time to obtain ethanolic Rizhoma Curcumae Longae extracts; combing and filtering the ethanolic Rizhoma Curcumae Longae extracts; concentrating the filtered ethanolic Rizhoma Curcumae Longae extracts by vacuum evaporation to a concentration of 100% (w/v) to produce 37.5 ml of an Extract 3a; and combining Extracts 1, 2, 2a, 3, and 3a to obtain the formulation. In additional embodiments, at least one of the extracts can be subjected to a purification process, such as, for example, a passage through a polymeric adsorption resin. The method can also involve reducing in size Caulis Sinomenii 4 parts, Radix Aconiti Lateralis Preparata 3 parts, Radix Paeoniae Alba 5 parts, Cortex Moutan 3 parts, and Rizhoma Curcumae Longae 2 parts; obtaining an aqueous extract of Caulis Sinomenii as an Extract 1; obtaining an alcoholic extract of Radix Aconiti Lateralis Preparata and Radix Paeoniae Alba as an Extract 2; obtaining a supercritical fluid extract of Cortex Moutan as an Extract 3; obtaining an alcoholic extract of Cortex Moutan as an Extract 4; obtaining a supercritical fluid extract of Rizhoma Curcumae Longae as an Extract 5; obtaining an alcoholic extract of Rizhoma Curcumae Longae as an Extract 6; and mixing the extracts with suitable excipients to obtain the formulation. The step of obtaining an aqueous extract of Caulis Sinomenii step can also involve soaking Caulis Sinomenii with water; refluxing soaked Caulis Sinomenii in water more than once to form aqueous extracts; combining and filtering the aqueous extracts; concentrating the filtered aqueous extract by vacuum evaporation to a relative density of 1.10˜1.11; cooling the concentrated extract; adding egg white to the cooled concentrated extract to form an egg white mixture; boiling the egg white mixture; centrifuging the egg white mixture; removing the supernatant of the egg white mixture; concentrating the supernatant by vacuum evaporation to a relative density of 1.13˜1.15 to form a concentrated filtrate; adding alcohol to the concentrated filtrate to form an alcoholic mixture with an alcohol content of about 60% (by volume); allowing the alcoholic mixture to stand unheated for sufficient time to form a liquid fraction and a solid fraction; filtering the liquid fraction; concentrating the filtered liquid fraction by vacuum evaporation to a relative density of 1.13˜1.15; adding β-cyclodextrin to the concentrated liquid fraction; adjusting the relative density of the concentrated liquid fraction to 1.10˜1.11; spray-drying the concentrated liquid fraction to produce Extract 1. The step of obtaining an alcoholic extract of Radix Aconiti Lateralis Preparata and Radix Peioniae Alba can involve, for example, soaking Radix Aconiti Lateralis Preparata and Radix Paeoniae Alba with alcohol; refluxing soaked Radix Aconiti Lateralis Preparata and Radix Paeoniae Alba in alcohol more than once to form an alcoholic extract; combining and filtering the alcoholic extract; concentrating the alcoholic extract by vacuum evaporation to a relative density of 1.13˜1.15; adjusting the relative density of the concentrated alcoholic extract to a relative density of 1.10˜1.11; spray-drying the concentrated alcoholic extract to produce Extract 2. The step of obtaining a supercritical fluid extract of Cortex Moutan can involve, for example, extracting Cortex Moutan with supercritical carbon dioxide to produce Extract 3. The step of obtaining an alcoholic extract of Cortex Moutan (Extract 4) can involve, for example, soaking the Cortex Moutan residue with alcohol; refluxing soaked Cortex Moutan in alcohol more than once to form an alcoholic extract; filtering the alcoholic extract; concentrating the filtered alcoholic extract by vacuum evaporation to a relative density of 1.09˜1.11; cooling the concentrated alcoholic extract; adding egg white to the cooled concentrated alcoholic extract to form an egg white mixture; boiling the egg white mixture; centrifuging the egg white mixture; filtering the supernatant from the centrifuged egg white mixture; concentrating the filtered supernatant by vacuum evaporation to a relative density of 1.13˜1.15; adding β-cyclodextrin to the concentrated supernatant to form a mixture; adjusting the relative density of the mixture to 1.10˜1.11; and spray-drying the concentrated mixture to produce Extract 4. The step of obtaining a supercritical fluid extract of Rizhoma Curcumae Longae (Extract 5) can involve, for example, extracting Rhizoma Curcumae Longae with supercritical carbon dioxide to produce Extract 5. The step of obtaining an alcoholic extract of Rizhoma Curcumae Longae (Extract 6) can involve, for example, soaking the Rizhoma Curcumae Longae residue with alcohol; refluxing the soaked Rizhoma Curcumae Longae residue in the alcohol more than once to obtain an alcoholic extract; filtering the alcoholic extract; concentrating the filtered alcoholic extract by vacuum evaporation to a relative density of 1.20; adding to the concentrated alcoholic extract; and drying the concentrated alcoholic extract by vacuum-drying method to produce Extract 6. The step of mixing the extracts with suitable excipients can involve, for example, mixing Extracts 1, 2, 3, 4, 5, and 6 with suitable excipients to form a powder mixture; granulating the powder mixture to form a granule mixture; and filling capsules with the capsule mixture and suitable excipients.

Additional embodiments of the invention provide a formulation for treating arthritis, the formulation having Caulis Sinomenii, Radix Aconitii Lateralis Preparata, Radix Paeoniae Alba, Cortex Moutan and Rhizoma Curcumae Longae; the formulation further obtained by a method having the steps of reducing the size of Caulis Sinomenii, Radix Aconitii Lateralis Preparata, Radix Paeoniae Alba, Cortex Moutan and Rhizoma Curcumae Longae; extracting Caulis Sincomenii, Radix Aconitii Lateralis Preparata, Radix Paeoniae Alba, Cortex Moutan and Rhizoma Curcumae Longae with suitable liquids to form extracts; concentrating the extracts; and combining the above extracts, where the Caulis Sinomenii, Radix Aconitii Lateralis Preparata, Radix Paeoniae Alba, Cortex Moutan and Rhizoma Curcumae Longae are in the proportion of (2-10:1-6:3-15:1-8:1-8) parts by weight respectively. The extracting step can also involve, for example, extracting Caulis Sinomenii, Radix Aconitii Lateralis Preparata, Radix Paeoniae Alba, together with an alcohol to produce an Extract 1; extracting Cortex Moutan with supercritical carbon dioxide to produce an Extract 2; and extracting Rhizoma Curcumae Longae with supercritical carbon dioxide to produce an Extract 3. The extracting Cortex Moutan step can be followed, for example, by extracting the Cortex Moutan residue with an alcohol to produce another extract. The method can also involve a step of subjecting at least one of the extracts to a purification process, such as, for example, passage through a polymeric adsorption resin. The formulation can be used, for example, as a method of treating arthritis in a mammal, by administering a therapeutically effective amount of the formulation. The form of arthritis to be treated can be, for example, rheumatoid arthritis or ankylosing spondylitis. The mammal can be, for example, a human.

Additional embodiments of the invention provide a method of manufacturing a medicament for the therapeutic and/or prophylactic treatment of arthritis and similar conditions, using a formulation having extracts of Caulis Sinomenii, Radix Aconitii Lateralis Preparata, Radix Paeoniae Alba, Cortex Moutan and Rhizoma Curcumae Longae. The separation technique can be, for example, thin layer chromatography, high performance liquid chromatography or a combination thereof. The comparing step can involve, for example, qualitative comparisons and quantitative comparisons.

Further embodiments of the present invention provide a formulation having about 5 parts of Caulis Sinomeni extract, 3 parts of Radix Aconitii Lateralis Preparata extract, 6 parts of Radix Paeoniae Alba extract, 3 parts of Cortex Moutan extract, and 3 parts of Rhizoma Curcumae Longae extract.

### BRIEF DESCRIPTION OF THE DRAWINGS

FIGS. 1A and 1B show the HPLC chromatograms for sinomenine reference and sinomenine in Mixture Dp respectively.

FIGS. 2A and 2B show the HPLC chromatograms for paeoniflorin reference and paeoniflorin in Mixture Dp respectively.

FIGS. 3A and 3B show the HPLC chromatograms for curcumin reference and curcumin in Mixture Dp respectively.

FIG. 4 show the TLC chromatograms for sinomenine reference and sinomenine in the composition respectively.

FIG. 5 show the TLC chromatograms for paeoniflorin reference and paeoniflorin in the composition respectively.

FIG. 6 show the TLC chromatograms for hypaconitine reference and hypaconitine in the composition respectively.

FIG. 7 show the TLC chromatograms for paeonol reference and paeonol in the composition-respectively.

FIG. 8 show the TLC chromatograms for curcumin reference and curcumin in the composition respectively.

FIG. 9A shows the HPLC chromatograms for the composition prepared without Radix Aconiti Lateralis Preparata, while FIGS. 9B and 9C show the HPLC chromatograms for aconitine, mesaconitine, and hypaconitine references and aconitine, mesaconitine, and hypaconitine in the composition respectively.

FIG. 10A shows the HPLC chromatograms for the composition prepared without Caulis Sinomenii, while FIGS. 10B and 10C show the HPLC chromatograms for sinomenine references and sinomenine in the composition respectively.

FIG. 11A shows the HPLC chromatograms for the composition prepared without Radix Paeoniae Alba, while FIGS. 11B and 11C show the HPLC chromatograms for paeoniflorin references and paeoniflorin in the composition respectively.

FIG. 12A shows the HPLC chromatograms for the composition that prepared without Cortex Moutan, while FIGS. 12B and 12C show the HPLC chromatograms for paeonol references and paeonol in the composition respectively.

FIG. 13A shows the HPLC chromatograms for the composition prepared without Rhizoma Curcumae Longae, while FIGS. 13B and 13C show the HPLC chromatograms for curcumin references and curcumin in the composition respectively.

FIGS. 14A, 14B and 14C are line graphs showing the anti-arthritic effects of Mixture Dp in adjuvant-induced arthritis on the parameters of paw volume, arthritic score and body weight respectively.

FIGS. 15A, 15B and 15C are line graphs demonstrating the anti-arthritic effects of the capsule obtained by the Encapsulation Process in adjuvant-induced arthritis on the parameters of paw volume, arthritic score and body weight respectively.

FIGS. 16A, 16B and 16C are line graphs showing the anti-arthritic effects of the capsule obtained by the Encapsulation Process in collagen II-induced arthritis on the parameters of paw volume, arthritic score and body weight respectively.

FIG. 17 is a graph showing the effect long term administration of the capsule obtained by the Encapsulation Process to the bodyweight of normal SD rats.

### DETAILED DESCRIPTION

As used in the present specification and claims, the terms “comprise,” “comprises,” and “comprising” mean “including, but not necessarily limited to”. For example, a method, apparatus, molecule or other item which contains A, B, and C may be accurately said to comprise A and B. Likewise, a method, apparatus, molecule or other item which “comprises A and B” may include any number of additional steps, components, atoms or other items as well. The term “consisting essentially of” means a narrower view of the term “comprising” wherein the number of additional steps, components, atoms or other items are more restricted, for example, to not more than a few additional of such steps or items. The term “consisting of” means that the method, apparatus, molecule, or other item which contains A, B and C is restricted to only A, B and C.

Also, unless otherwise defined, all technical and scientific terms used herein have the same meaning as commonly understood by one of ordinary skill in the art to which this invention belongs.

General Description

All herbal components used in experimental formulation and studies of the present invention were identified according to the exacting standards and requirements of the Pharmacopoeia of the People's Republic of China (2000 Edition, Volume I) for each herb. Both morphological and microscopic examinations, as well as chemical analysis were performed where monographic data were available. The botanical and herbal terminology used herein are the same as that used in the Pharmacopoeia of the People's Republic of China (2000 Edition, Volume I) and should be interpreted according to this publication. In this text, the terms “plant materials” or “herbal materials” refer to the herbs and the various parts or tissues of the herbs used in the invention. These terms are used interchangeably.

1. Components of the Formulation of the Present Invention

1.1 Components

In some embodiments of the inventoin, a formulation comprising the five plant components elow is prepared.

Component 1 is obtained from the herbs Sinomenium acutum (Thumb.) Rehd. et Wils or Sinomenium acutum (Thumb.) Rehd. et Wils. var. cinereum Rehd. et Wils of the genus Sinomenium.

Component 2 is the herb Aconitum carmichaeli Debx.

Component 3 is the herb Paeonia lactiflora Pall.

Component 4 is the herb Paeonia suffruticosa Andr.

Component 5 is the herb Curcumae longae L.

Specifically, the plant materials are:

Component 1 is Caulis Sinomenii or the main stem of the herbs Sinomenium acutum (Thumb.) Rehd. et Wils or Sinomenium acutum (Thumb.) Rehd. et Wils. var. cinereum Rehd. et Wils.

Component 2 is Radix Aconiti Lateralis Preparata, the lateral roots of the herb Aconitum carmichaeli Debx.

Component 3 is Radix Paeoniae Alba, the roots of the herb Paeonia lactiflora Pall.

Component 4 is Cortex Moutan, the bark of the peony herb Paeonia suffruticosa Andr.

Component 5 is Rhizoma Curcumae Longae, the rhizome of the herb Curcumae longae L.

Caulis Sinomenii

Caulis Sinomenii is the dried Caulis of Sinomenium acutum (Thunb.) Rehd. et Wils. or Sinomenium acutum (Thunb.) et Wils. var. cinereum Rehd. et. Wils. Sinomenium cultivated from nationwide for use of Chinese materia medica.

Radix Aconiti Lateralis Preparata

Radix Aconiti Lateralis Preparata is the prepared lateral root of the herb Aconitum carmichaeli Debx. Plants of the genus Aconiti produce various kinds of C₁₉ norditerpenoids and C₂₀ diterpenoids. Among these chemicals, some 8-acetyl-14-aroyl diester C₁₉ alkaloids such as aconitine, are highly toxic. The alkaloid components and their concentrations vary with the species, place of origin time of harvest and method of preparation. Despite their toxicity, aconite roots have been used as important ingredients in traditional Chinese medicine for their pharmaceutical value. To render the roots safe for further use, they are generally prepared by boiling in water for a few hours before being dried for storage.

Radix Paeoniae Alba

Radix Paeoniae Alba (white Peony Root; Baishao), the dried peeled root of Paeonia lactiflora Pall, is one of the Chinese traditional tonic crude products. It is also been used as a spasmolytic and pain-relieving agent and has long been used to regulate the menstrual flow, for the treatment of menstrual disorders and to relieve abdominal spasmodic pain and muscle stiffness.

Cortex Moutan

Cortex Moutan, the bark of the stem and root of the peony herb Paeonia suffruticosa Andr of the paeoniaceae family. The dried product is called Moutan bark, tree peony bark or Mudanpi. It is usually harvested in the autumn and dried after the more fibrous parts are removed.

Rhizome Curcumae Longae

Rhizoma Curcumae Longae is an important ingredient of Chinese medical preparations and is also a coloring agent, and has been found to be a rich source of phenolic compounds, namely curcuminoids. Curcumin [1,7-bis (4-hydroxy-3-methoxyphenyl)-1,6-heptadiene-3,5,-dione], the principal natural yellow pigment, is widely used for the coloring of foods, for example, pickles and snacks.

1.2 Proportion of the Components

Preferably, the formulation comprises plant materials in the following proportion by dry weight: Caulis Sinomenii: about 2-10 parts; Radix Aconitii Lateralis Preparata: about 1-6 parts; Radix Paeoniae Alba: about 3-15 parts, Cortex Moutan: about 1-8 parts; and of and Rhizoma Curcumae Longae: about 1-8 parts.

2. Materials and Methods

2.1 Herbs

Caulis Sinomenii ( [symbol] ), a wild product of Anhui Province, was purchased from Zhong-Yue Herbal Pharmaceutical Union Company.

Radix Aconiti Lateralis Preparata ( [symbol] ), a product of Good Agricultural Practice (GAP) base of Radix Aconiti Lateralis Preparata at Jiangyou County of Chengdu City, and Rizhoma Curcumae Longae ( [symbol] ), a product of GAP base of Rizhoma Curcumae Longae at Shuangliu County of Chengdu City, were purchased from a pharmaceutical market at Chengdu City of Shichuan Province. Radix Paeoniae Alba ( [symbol] ), a product of GAP base of Radix Paeoniae Alba at Bozhou city of Anhui Province, and Cortex Moutan ( [symbol] ), a product of GAP base of Cortex Moutan at Tongling city of Anhui Province, were purchased from a pharmaceutical market at Bozhou City of Anhui Province.

The above herbs are obtained in their dried form as Chinese medicinal herbs are usually dried and sold as such. Accordingly, the weights given for the herbs of the present invention are the dried weights of the respective herbs.

2.2 Equipment

An RT-80 pulverizer (CERT-04, FARGO, Taiwan, China), a multifunction extraction cauldron for herbal medicine (DT-3m³, Wenzhou, China), an alcohol recovery distiller (ZWN-1000, Tianjing, China), and a supercritical carbon dioxide extraction apparatus set (HA421-40-96, Nantong, China) were used to prepare the different herbal extracts. A plethymometer (UGO Basile, Italy) and IITC 336 tail flick analgesia meter (IITC Inc, Woodland Hills, Calif., USA) were employed to evaluate the anti-inflammatory and anti-nociceptive activities of the various herbal extracts.

2.3 Extraction Methods

2.3.1 Generalized Extraction Method

The generalized extraction process comprises reducing the size of the herbal materials (for example by pulverizing) followed by extraction by refluxing with a suitable solvent.

The extracts are then concentrated and/or dried by vacuum rotary evaporation. The components of the present invention were subjected to various combinations of extraction methods by water, alcohol and supercritical carbon dioxide in order to determine the optimal process with the highest desired pharmacological effects. At any point in the process, the intermediate products of any step may be subjected to concentration, clarification or purification steps. Thereafter, some of the extracts were further purified and tested for improved efficacy.

2.3.2 Specific Extraction Methods

Process A to Obtain Mixture A

Caulis Sinomenii, Radix Aconiti Lateralis Preparata, Radix Paeoniae Alba, Cortex Moutan, and Rizhoma Curcumae Longae were pulverized to a coarse powder. Caulis Sinomenii, Radix Aconiti Lateralis Preparata and Radix Paeoniae Alba were refluxed together with 6× (w/w) water for a total of 3 times, 1 hour each. The water extracts were combined, filtered, and concentrated to the concentration of 100% (w/v) by vacuum evaporation (70° C., 0.08 Mpa) to produce extract A1. Next, Cortex Moutan and Rizhoma Curcumae Longae were extracted by supercritical fluid extraction (SFE) technique with supercritical carbon dioxide (21.7 L/h) to produce extracts A2 and A3, respectively. Mixture A was made by combining and mixing well the extracts A1, A2, and A3.

Process B to Obtain Mixture B

Caulis Sinomenii, Radix Aconiti Lateralis Preparata, Radix Paeoniae Alba, Cortex Moutan, and Rizhoma Curcumae Longae were pulverized to a coarse powder. Caulis Sinomenii, Radix Aconiti Lateralis Preparata and Radix Paeoniae Alba were refluxed together with 80% ethanol (4 times of the herb, w/w) for 3 times, 1 hour each. The ethanol extracts were combined, filtered, and concentrated to the concentration of 100% (w/v) by vacuum evaporation (70° C., 0.08 Mpa) to produce extract B1. Next, Cortex Moutan and Rizhoma Curcumae Longae were extracted by SFE with supercritical carbon dioxide (21.7 L/h) to produce extracts B2 and B3, respectively. Mixture B was made by combining and mixing the extracts B1, B2, and B3.

Process C to Obtain Mixture C

Caulis Sinomenii, Radix Aconiti Lateralis Preparata, Radix Paeoniae Alba, Cortex Moutan, and Rizhoma Curcumae Longae were pulverized to a coarse powder. Caulis Sinomenii, Radix Aconiti Lateralis Preparata and Radix Paeoniae Alba were refluxed 3 times with 6× water w/w, 1 hour each. The water extracts were combined, filtered, and concentrated to the concentration of 100% (w/v) by vacuum evaporation (70° C., 0.08 Mpa) to produce extract C1. Next, Cortex Moutan was extracted by SFE with supercritical carbon dioxide (21.7 L/h) to produce extract C2, the residue of Cortex Moutan after SFE was refluxed with 80% ethanol (4 times of the herb, w/w) 3 times, 1 hour each. The ethanol extracts were combined, filtered, and concentrated to the concentration of 100% (w/v) by vacuum rotary evaporation (70° C., 0.08 Mpa) to produce extract C3. Next Rizhoma Curcumae Longae was extracted by SFE with supercritical carbon dioxide (21.7 L/h) to produce extract C4, the residue of Rizhoma Curcumae Longae after SFE was refluxed with 80% ethanol (4 times of the herb, w/w) for 3 times, 1 hour each. The ethanol extracts were combined, filtered, and concentrated to the concentration of 100% (w/v) by vacuum rotatory evaporation (70° C., 0.08 Mpa) to produce extract C5. Mixture C was made by combining and mixing the extracts C1, C2, C3, C4, and C5.

Process D to Obtain Mixture D

Caulis Sinomenii, Radix Aconiti Lateralis Preparata, Radix Paeoniae Alba, Cortex Moutan, and Rizhoma Curcumae Longae were pulverized to a coarse powder. Caulis Sinomenii, Radix Aconiti Lateralis Preparata and Radix Paeoniae Alba were refluxed together with 80% ethanol (4 times of the herb, w/w) 3 times, 1 hour each. The ethanol extracts were combined, filtered, and concentrated to the concentration of 100% (w/v) by vacuum evaporation (70° C., 0.08 Mpa) to produce extract D1. Cortex Moutan was extracted by SFE with supercritical carbon dioxide (21.7 L/h) to produce extract D2, the residue of Cortex Moutan after SFE was refluxed with 80% ethanol (4 times of the herb, w/w) 3 times, 1 hour each. The ethanol extracts were combined, filtered, and concentrated to the concentration of 100% (w/v) by vacuum evaporation (70° C., 0.08 Mpa) to produce extract D3. Next Rizhoma Curcumae Longae was extracted by SFE with supercritical carbon dioxide (21.7 L/h) to produce extract D4, the residue of Rizhoma Curcumae Longae after SFE was refluxed with 80% ethanol (4 times of the herb, w/w) 3 times, 1 hour each. The ethanol extracts were combined, filtered, and concentrated to the concentration of 100% (w/v) by vacuum evaporation (70° C., 0.08 Mpa) to produce extract D5. Mixture D was made by combining and mixing well the extracts D1, D2, D3, D4, and D5.

Process E to Obtain Mixture E

Caulis Sinomenii, Radix Aconiti Lateralis Preparata, Radix Paeoniae Alba, Cortex Moutan, and Rizhoma Curcumae Longae were pulverized to a coarse powder. Caulis Sinomenii was extracted by soaking with 0.1% hydrochloric acid solution (pH 2.0, 6 times of the herb, w/w) 3 times, 12 hours each, at room temperature. The hydrochloric acid extracts were combined, filtered, and adjusted to pH 6.0 with 1% sodium hydroxide solution, then concentrated to the concentration of 100% (w/v) by vacuum evaporation (70° C., 0.08 Mpa) to produce extract E1. Next, Cortex Moutan and Rizhoma Curcumae Longae were extracted by SFE with supercritical carbon dioxide (21.7 L/h) to produce extract E2 and E3, respectively. Radix Aconiti Lateralis Preparata, Radix Paeoniae Alba, and the residue of Cortex Moutan after SFE were refluxed together with 80% ethanol (4 times of the herb, w/w) 3 times, 1 hour each, the ethanol extracts were combined, filtered, and (concentrated to the concentration of 100% (w/v)) fully dried by vacuum evaporation (70° C., 0.08 Mpa) to produce extract E4. The residue of Rizhoma Curcumae Longae after SFE was further extracted by soaking with 0.1% hydrochloride solution (pH=10, 6 times of the herb, w/w) 3 times, 1 hour each, the alkaloid extracts thus obtained were combined, filtered, and adjusted to pH 4.0 with 1% hydrochloric acid solution, then concentrated to a concentration of 100% (w/v) by vacuum evaporation (70° C., 0.08 Mpa) to produce extract E5. Mixture E was made by combining and mixing well the extracts E1, E2, E3, E4, and E5.

Process F to Obtain Mixture F

Caulis Sinomenii, Radix Aconiti Lateralis Preparata, Radix Paeoniae Alba, Cortex Moutan, and Rizhoma Curcumae Longae were pulverized to a coarse powder, then refluxed together with water (6 times of the herb, w/w) 3 times, 1 hour each. The water extracts were combined, filtered, and concentrated to the concentration of 100% (w/v) by vacuum evaporation (70° C., 0.08 Mpa) to produce Mixture F, an aqueous extract of all five herbs.

Process G to Obtain Mixture G

Caulis Sinomenii, Radix Aconiti Lateralis Preparata, Radix Paeoniae Alba, Cortex Moutan, and Rizhoma Curcumae Longae were pulverized to a coarse powder, then refluxed together with ethanol (4 times of the herb, w/w) 3 times, 1 hour each. The ethanol extracts were combined, filtered, and concentrated to the concentration of 100% (w/v) by vacuum evaporation (70° C., 0.08 Mpa) to produce Mixture G, an alcoholic extract of all five herbs.

2.3.3 Purification Process

An extract of the formulation obtained from any of the various possible processes described above may be further purified. One way of increasing the purity of the desired is to subject the extract to one or more separation techniques in which the unwanted components are reduced, thus increasing the relative abundance of the desired components in the extract.

A preferred separation technique is to use a polymeric adsorption resin that preferentially binds the desired components but not the unwanted components under certain conditions. Thereafter, these conditions (eg pH, polarity of eluting solvent) are changed to release the bound components.

2.3.4 Encapsulation Process

The general steps below describe how the extract of the present invention can be packaged as a capsule for oral administration. The extract is first precipitated and then used to fill capsule shells. Another method that can be used is to co-precipitate the extracts, either individually or with other extracts, with suitable excipients, before filling capsule shells.

2.3.5 First Implementation Example

A first implementation example of the invention uses Process D to obtain the extract followed by an example of a purification process to illustrate how the invention may be practiced.

The formulation of the first implementation comprises the herbs in the following proportion by weight: Caulis Sinomenii 5 parts, Radix Aconiti Lateralis Preparata 3 parts, Radix Paeoniae Alba 6 parts, Cortex Moutan 3 parts, and Rizhoma Curcumae Longae 3 parts.

The following procedure is for the preparation of an extract in the proportion of the formulation of the first implementation. The five dried herbs were separately pulverized into coarse powders.

Caulis Sinomenii 62.5 g, Radix Aconiti Lateralis Preparata 37.5 g and Radix Paeoniae Alba 75 g were pulverized and refluxed together with 1250 ml 80% ethanol for 3 times, 1 hour for each time. The ethanolic extracts were then combined and filtered. The ethanolic filtrate was then concentrated by vacuum evaporation (70° C., 0.08 Mpa) to the concentration of 100% (w/v) to produce 175 ml of Extract 1 (ie, a total of 175 g of herbs extracted in a final volume of 175 ml of Extract 1).

Cortex Moutan 37.5 g was extracted by supercritical fluid extraction with supercritical carbon dioxide (21.7 L/h) to produce 0.8 g of Extract 2. The residue after SFE was then refluxed with 187.5 ml of 80% ethanol for 3 times, 1 hour for each time. The ethanolic extracts were combined and filtered. The ethanolic filtrate was then concentrated by rotary vacuum evaporation (70° C., 0.08 Mpa) to the concentration of 100% (w/v) to produce 37.5 ml of Extract 3.

Similarly, Rizhoma Curcumae Longae 37.5 g was extracted by supercritical extraction with supercritical carbon dioxide (21.7 L/h) to produce 2.8 ml of Extract 4. The residue after SFE was then refluxed with 187.5 ml of 80% ethanol for 3 times, 1 hour for each time. The ethanolic extracts were combined and filtered. The ethanolic filtrate was then concentrated by vacuum evaporation (70° C., 0.08 Mpa) to the concentration of 100% (w/v) to produce 37.5 ml of Extract 5.

Extracts 1, 3 and 5 were combined and further concentrated to dryness by rotary evaporation (70° C., 0.08 Mpa) and then dissolved in 40 ml of 80% ethanol. The ethanolic solution was loaded onto an Amberlite XAD-7HP polymeric resin (Rohm and Haas Company, USA.) column (100×5 cm, the resin height was 65 cm), which has been pretreated by flushing with 5000 ml distilled water, 2000 ml 95% ethanol and 2000 ml distilled water in sequence, and then eluted with 1900 ml of 5% ethanol, 3000 ml of 40% ethanol and 3000 ml of 80% ethanol in sequence. The eluates of both 40% ethanol and 80% ethanol were collected, combined and then concentrated to a volume of 500 ml by rotary vacuum evaporation (70° C., 0.08 Mpa), and further lyophilized to obtain 13.1 g of a purified fraction. A mixture (16.1 g) was obtained by mixing this purified fraction (13.1 g) with Extract 2 (0.9 g) and Extract 4 (2.67 ml). As this mixture was obtained by Process D and was subsequently purified, this mixture thus obtained may be termed Mixture Dp and will be referred to Mixture Dp in the other sections of the description.

2.3.6 Second Implementation Example

A second implementation example of the invention illustrates how the extraction steps for the herbal components can incorporate inclusion of suitable excipients to form capsules as an example of how the formulation may be further processed into a form suitable for oral administration.

A formulation of a second implementation comprises herbs in the following proportion by weight: Caulis Sinomenii 4 parts, Radix Aconiti Lateralis Preparata 3 parts, Radix Paeoniae Alba 5 parts, Cortex Moutan 3 parts, and Rizhoma Curcumae Longae 2 parts. The following procedure describes the preparation of an extract in the proportion of the formulation of the second implementation.

The five herbs were pulverized into coarse powders. Caulis Sinomenii 720 g was soaked with 5760 ml water for 0.5 hour and refluxed for 3 times, 1.5 hours for each time. The water extracts were then combined and filtered. The filtrate was then concentrated by vacuum evaporation (70° C., 0.08 Mpa) to a relative density of 1.10˜1.11 (70° C.). After the temperature of the concentrated filtrate has cooled to room temperature, 7.2 g of egg white was added, mixed well and the mixture boiled to clarify the concentrated extract of Caulis Sinomenii. The mixture was then centrifuged to separate the liquid component as a supernatant from the solid component. The filtered supernatant was concentrated by vacuum evaporation (70° C., 0.08 Mpa) to a relative density of 1.13˜1.15 (70° C.). Alcohol was added to the concentrated filtrate with continuous mixing until the alcohol content reached 63% (by volume). The mixture left to stand for 24 hours at room temperature to allow sedimentation of the mixture. The upper liquid portion or supernatant was then removed, filtered and concentrated by vacuum evaporation (70° C., 0.08 Mpa) to a relative density of 1.13˜1.15 (70° C.). The concentrated filtrate, after addition of 12.6 g of β-cyclodextrin and the relative density was adjusted to 1.10˜1.11 (70° C.), was dried by spray-drying method (inlet temperature 140° C., outlet temperature 100° C.) to produce 65.7 g of Extract 1.

Radix Aconiti Lateralis Preparata 540 g and Radix Paeoniae Alba 900 g together were soaked with 7200 ml 80% ethanol for 0.5 hour and then refluxed for 3 times, 1.5 hours for each time. The ethanolic extracts were then combined and filtered. The ethanolic filtrate was then concentrated by vacuum evaporation (70° C., 0.08 Mpa) to the relative density of 1.13˜1.15 (70° C.). The concentrated filtrate, after the relative density was adjusted to 1.10˜1.11 (70° C.), was dried by spray-drying method (inlet temperature 140° C., outlet temperature 100° C.) to produce 175.1 g of Extract 2.

Cortex Moutan 520 g was pulverized and extracted by supercritical fluid extraction with supercritical carbon dioxide (250 L/h) to produce 11.6 g of Extract 3. The residue after SFE was soaked with 2600 ml of 80% ethanol for 0.5 hour and then refluxed for 3 times, 1 hour for each time. The alcoholic filtrate was then concentrated by vacuum evaporation (70° C., 0.08 Mpa) to a relative density of 1.09˜1.11 (70° C.). After the temperature of concentrated filtrate reached room temperature, 3.9 g of egg white was added, mixed well and boiled. The mixture was then centrifuged to separate the liquid component as a supernatant from the solid component. The filtrated supernatant was concentrated by vacuum evaporation (70° C., 0.08 Mpa) to a relative density of 1.13˜1.15 (70° C.). The concentrated filtrate, after addition of 9.5 g of β-cyclodextrin and the relative density was adjusted to 1.10˜1.11 (70° C.), was dried by spray-drying method to produce 49.1 g of Extract 4.

Rizhoma Curcumae Longae 360 g was pulverized and extracted by supercritical fluid extraction with supercritical carbon dioxide (250 L/h) to produce 26.7 ml of Extract 5. The residue after SFE was soaked with 2160 ml of 80% ethanol for 0.5 hour and then refluxed for 3 times, 1 hour for each time. The alcoholic filtrate was then concentrated by vacuum evaporation (70° C., 0.08 Mpa) to a relative density of 1.20 (70° C.). The concentrated filtrate, after addition of 18.0 g of starch, was dried by vacuum-drying method (70° C., 0.09 Mpa) to produce 37.3 g of Extract 6.

After addition and mixing well with 100 g of β-cyclodextrin, Extracts 3 (11.6 g) and 5 (26.7 ml) were mixed well with Extracts 1 (65.7 g), 2 (175.1 g), 4 (49.1 g), and 6 (37.3 g), and 15.3 g of carboxymethyl starch sodium and the balance with starch to make the total amount of the powder mixture to 510 g. This mixture was then granulated. Each No. 0 capsules may be filled with the granules at 0.51 g/each, together with 10.2 g of carboxymethyl starch sodium and 1.5 g magnesium stearate.

The extract obtained by this implementation was used in the assay tests described in the Quality Control Section below.

2.4 Comparison of Processes A-G

The two criteria for determining the optimal extraction process from among processes A-G described above are maximal extractions of known active ingredients and maximal beneficial pharmacological effects.

The quantification of the known main active components in the herbal extracts obtained by the various processes was determined by high performance liquid chromatography (HPLC). The known components of the herbs used in the present invention include sinomenine, paeoniflorin, paeonol and curcumin.

For use as reference standards, purified sinomenine, paeoniflorin, paeonol and curcumin were purchased from National institute for the Control of Pharmaceutical and Biological Products of the People's Republic of China. Demethoxycurcumin and bisdemethoxycurcumin were prepared and verified in our own laboratory. Acetonitrile, acetic acids, triethylamine (HPLC grade) were obtained from Merck (Darmstadt, Germany) and used as received.

The HPLC system used was a Agilent quaternary HPLC model HP 1100 series (Hewlett-Packard, Palo Alto, Calif.), fitted with Altima C₁₈ column (250×4.5 mm, 5 μm) and coupled a DAD detector. A Millipore Swinnex type filter (pore size=0.45 μm) was obtained from Millipore (Millipore, Hong Kong).

The standard solutions used were obtained by dissolving accurate amount of sinomenine, paeoniflorin, paeonol and curcumin in methanol respectively to produce a solution with a concentration of 0.2˜0.5 mg/ml for each compound.

To prepare the herbal extracts for HPLC analysis, 20 ml of methanol was added to 0.1 g of the dried extracts in a conical flask, and the flask and contents weighed. The mixture was then sonicated for 20 minutes. The mixture was cooled, weigh again and any loss in weight was compensated with methanol before being vigorously shaken and filtered through a 0.21 μm nylon syringe filter to obtain the test solutions.

The mobile phase used for above chemicals was acetonitrile-phosphate buffer (pH=7.4), filtered through a 0.451 μm Millipore filter and degassed prior to use, in a gradient mode in which the ratio of acetonitrile at 0, 30, 40, and 50 min was 10%, 90%, 10%, and 10%, respectively. The flow-rate was maintained at 0.8 ml/min, the detection of sinomenine, paeoniflorin, paeonol and curcumins was performed at wavelengths of 262 nm, 230 nm, 270 nm and 430 nm, respectively, under a constant temperature (25±0.1° C.).

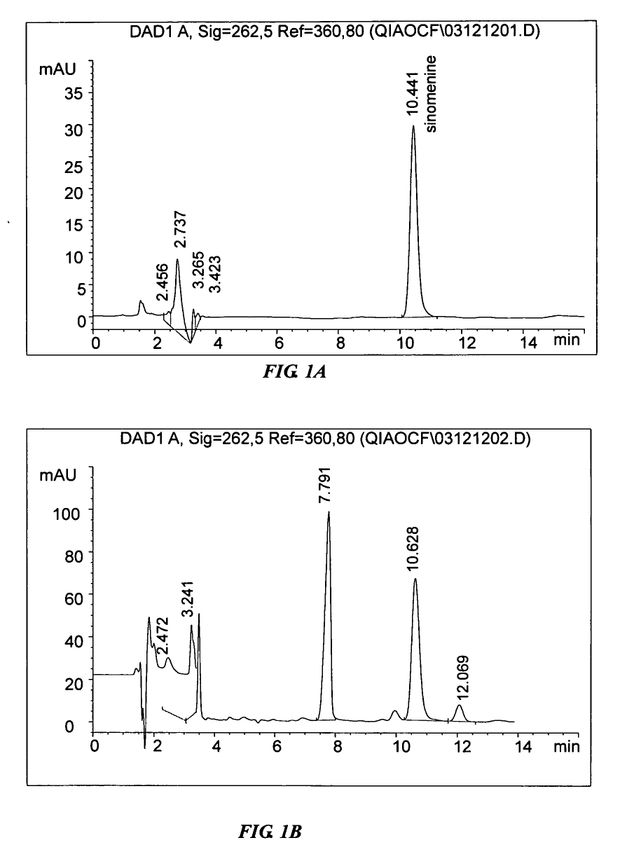

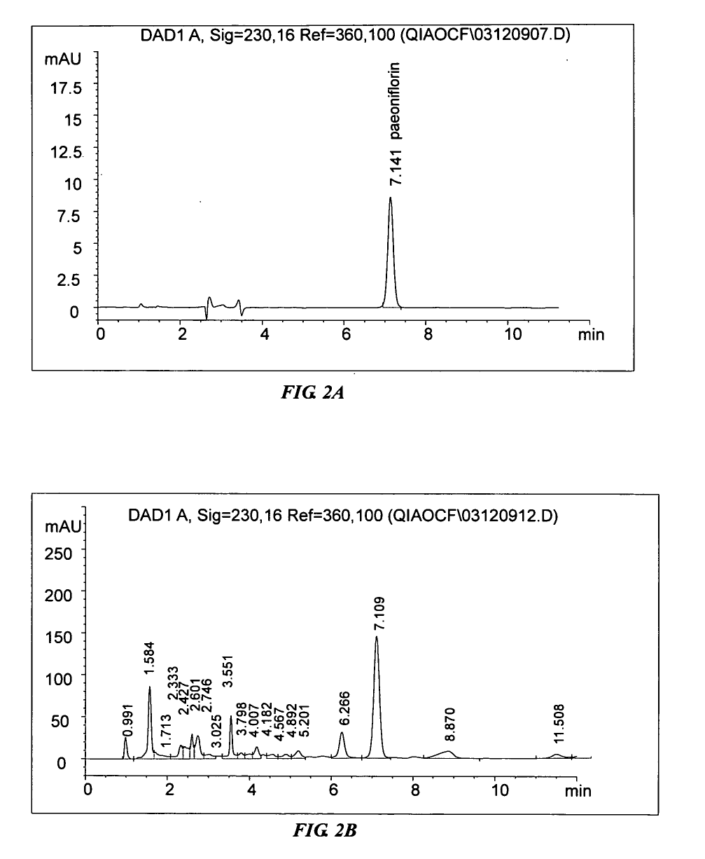

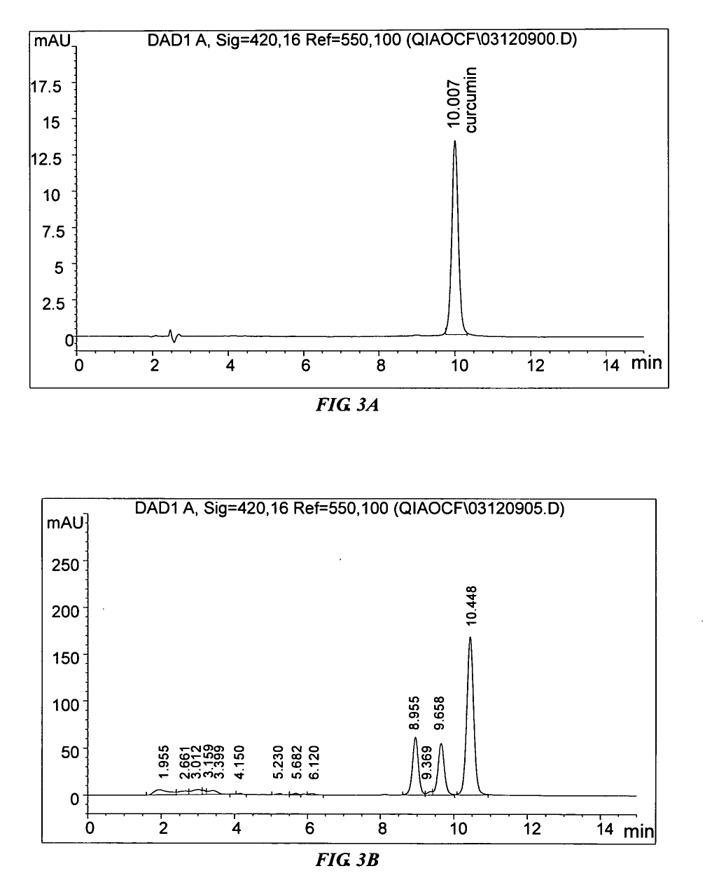

Results may be seen in the examples shown for the Mixture Dp in FIGS. 1-3. FIGS. 1A and 1B show the HPLC chromatograms for sinomenine reference and sinomenine in Mixture Dp respectively; FIGS. 2A and 2B show the HPLC chromatograms for paeoniflorin reference and paeoniflorin in Mixture Dp respectively, while FIGS. 3A and 3B show the HPLC chromatograms for curcumin reference and curcumin in Mixture Dp respectively.

2.5 Quality Control Standards.

For quality control, the following assay tests were used to detect the presence of some of the usable markers or major active ingredients (sinomenine, paeoniflorin, paeonol, curcumin, hypaconitine, aconitine, mesaconitine and hypaconitine) of the formulation. While these tests were applied to the formulation for packaging in capsule form as obtained by the Encapsulation Process above, they can also be applied to the extracts produced from Process A-G and Mixture Dp.

By making use of the combination of assay tests below, verification of the combination of plant materials of the present invention can be done.

2.5.1 Assay Test No 1. Identification of Caulis Sinomenii

Ten ml of ethyl alcohol was added to 1 g of the extract obtained from the Encapsulation Process and the mixture sonicated for 30 minutes, and then filtered. Filtrate was evaporated to dryness under a stream of air to obtain a residue. Ethyl alcohol (1 ml) was added to dissolve the residue so as to make up the sample or test solution.

Separately, 0.3 g of Caulis Sinomenii powder was similarly used to prepare a reference solution by addition of ethy alcohol. In addition, produce one or more reference samples were also made by dissolving sinomenine in ethyl alcohol (1 ml:0.5 mg).

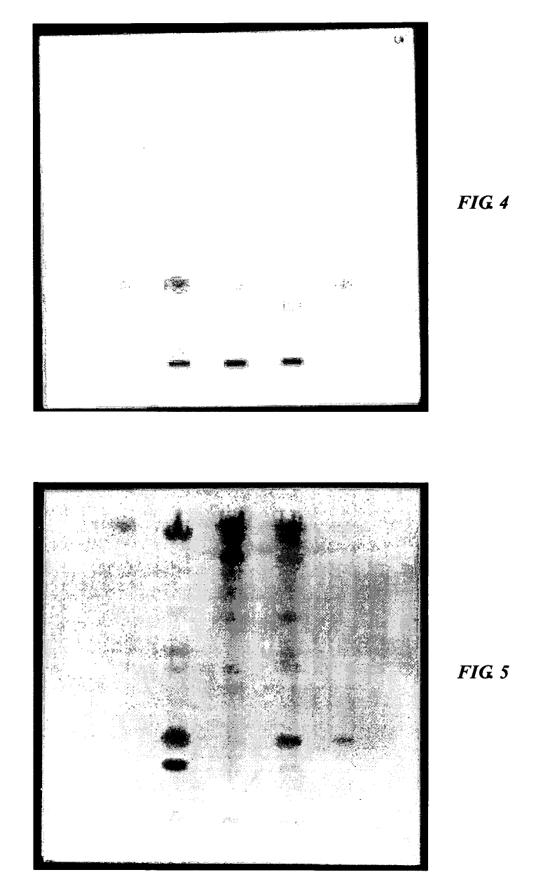

According to the Chromatography Procedure on page 35 of the Appendices of Chinese Pharmacopoeia (Volume One VI B, 2000 edition), 5 μL of the sample and the two reference solutions were separately loaded onto the same plate of silica gel G for thin-layer chromatography (TLC) analysis as were done for the other assays. A mixture of toluene, ethyl acetate, methanol and triethylamine (7:2:0.5:05) was used as the solvent to separate the samples. After separation, the plate was removed and air dried, and then sprayed with a dilute solution of potassium bismuth iodide to develop and visualize the chromatogram. The chromatogram produced by the sample solution showed the same orange-yellow spots as that displayed by each reference solution in their respective areas. This showed that the active ingredient/marker sinomenine was present in the extract obtained from the Encapsulation Process. Results may be seen in the examples shown for the composition in FIG. 4.

2.5.2 Assay Test No 2 Identification of Radix Paeoniae Alba

Ten ml of ethyl alcohol was added to 1 g of the extract obtained from the Encapsulation Process and the mixture sonicated for 30 minutes, and the solution filtered. The filtrate was evaporated to dryness under a stream of air to obtain a residue. One ml ethyl alcohol was then added to dissolve the residue so as to make up the sample or test solution.

Separately, 0.3 g of Radix Paeoniae Alba powder was used to prepare a reference solution by the same procedure. In addition, a reference sample was prepared by adding paeoniflorin to ethyl alcohol (1 ml:1 mg).

According to the Chromatography Procedure on page 35 of the Appendices of Chinese Pharmacopoeia (Volume One VI B, 2000 edition), 5 μL of the sample solution and one 5 μL each of the two reference solutions were separately loaded onto the same TLC plate of silica gel G. A mixture of chloroform, ethyl acetate, methanol and formic acid (40:5:10:0.2) was used as the solvent to separate the samples. After separation, the plate was removed and air dried, and sprayed with a dilute solution of vanillin sulfuric acid, and heated for 5 minutes in 105° C. in an oven to develop and visualize the chromatogram. The chromatogram produced by the sample solution showed the same blue-black spots as that displayed by the two reference solutions in their respective areas. This showed that the active ingredient/marker paeoniflorin was present in the extract obtained from the Encapsulation Process. Results may be seen in the examples shown for the composition in FIG. 5.

2.5.3 Assay Test No 3 Identification of Radix Aconitii Lateralis Preparata

Hydrochloric acid (30 ml, 0.05 mol/L) was added to 3.5 g of the extract obtained from the Encapsulation Process and the mixture was sonicated for 30 minutes and then extracted three times with 15 ml of ethyl acetate each time. The aqueous solution was then extracted with chloroform three times using 15 ml of chloroform each time. The extracts recovered from the chloroform solvent were combined and evaporated to dryness under a stream of air to obtain a residue. The residue recovered was dissolved in 1 ml of hydrochloric acid (0.01 mol/L) so as to make up the sample or test solution.

Separately, 1.5 g of Radix Aconiti Lateralis Preparata powder was used to prepare a reference solution by the same procedure. In addition, produce one more reference sample by adding hypaconitine to hydrochloride acid (0.01 mol/L) (1 ml:1 mg).

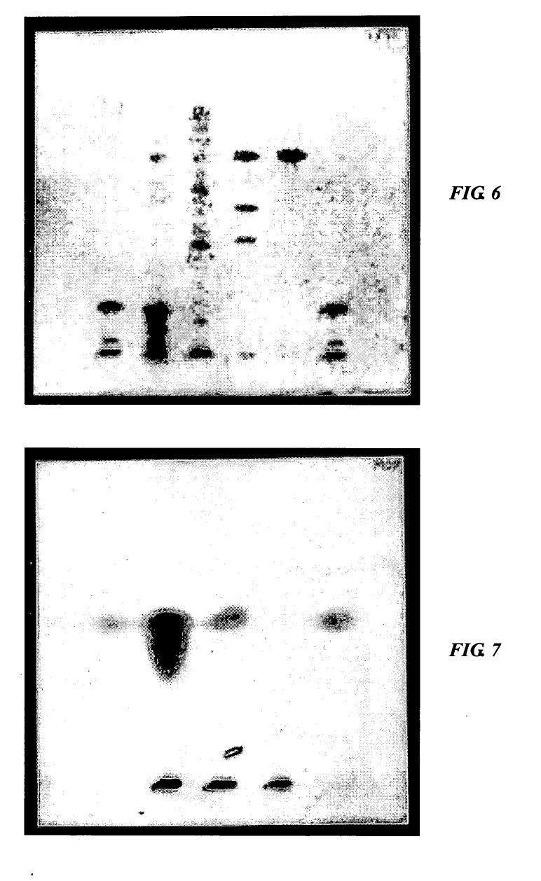

According to the Chromatography Procedure on page 35 of the Appendices of Chinese Pharmacopoeia (Volume One VI B, 2000 edition), 20 μL of the sample solution and 10 μL each of the reference solutions were loaded onto the same TLC plate of silica gel G. A mixture of toluene, ethyl acetate, and triethylamine (8:4:1) was used to separate the samples. Thereafter, the plate was removed, air dried, and sprayed with a dilute solution of potassium bismuth iodide to develop and visualize the chromatogram. The chromatogram produced by the sample solution showed the same orange-yellow spots as that displayed by each reference solution in their corresponding areas. This showed that the active ingredient/marker hypaconitine was present in the extract obtained from the Encapsulation Process. Results may be seen in the examples shown for the composition in FIG. 6.

2.5.4 Assay Test No 4 Identification of Cortex Moutan

Ten ml of ethyl alcohol was added to 1 g of the extract obtained from the Encapsulation Process and the mixture was sonicated for 30 minutes, and then filtered. The filtrate was evaporated to dryness under a stream of air. One ml ethyl alcohol was then added to dissolve the residue so as to make up the sample or test solution.

Separately, 0.3 g of Cortex Moutan powder was used to prepare a reference solution by the same procedure. In addition, one more reference sample by produced by addition of paeonol to ethyl alcohol (1 ml:1 mg).

According to the Chromatography Procedure on page 35 of the Appendices of Chinese Pharmacopoeia (Volume One VI B, 2000 edition), 5 μL each of the sample and two reference solutions were loaded onto the same TLC plate of silica gel G. A mixture of hexane, ethyl acetate, and formic acid (10:2:0.5:05) as the solvent system was used to separate the components. Thereafter, the plate was removed, air dried, and sprayed with an ethanolic solution of ferric chloride, and heated 5 minutes at 105° C. in an oven to develop and visualize the chromatogram. The chromatogram produced by the sample solution showed the same black spots as that displayed by each reference solution in their corresponding areas. This showed that the active ingredient/marker paeonol was present in the extract obtained from the Encapsulation Process. Results may be seen in the examples shown for the composition in FIG. 7.

2.5.5 Assay Test No 5 Rhizoma Curcumae Longae

Ten ml of ethyl alcohol was added to 1 g of the extract obtained from the Encapsulation Process and the mixture was sonicated for 30 minutes, and then filtered. The filtrate was evaporated to dryness under a stream of air. One ml of ethyl alcohol was used to dissolve the residue so as to make up the sample or test solution.

Separately, 0.3 g of Rhizoma Curcumae Longae powder was used to prepare a reference solution by the same procedure. In addition, another reference sample was produced by adding curcumin to ethyl alcohol (1 ml:0.3 mg).

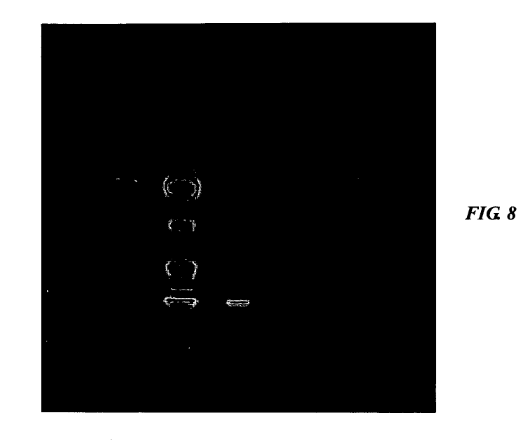

According to the Chromatography Procedure on page 35 of the Appendices of Chinese Pharmacopoeia (Volume One VI B, 2000 edition), 5 μL each of the sample and two reference solutions were loaded onto the same TLC plate of silica gel G. A mixture of chloroform, methanol, and formic acid (96:4:0.7) was used to separate the components. The plate was removed, air dried and viewed under an ultraviolet lamp (365 nm). The chromatogram produced by the sample solution showed the same yellow fluorescent spots as those shown by each of the reference solutions in their corresponding areas. This showed that the active ingredient/marker curcumin was present in the extract obtained from the Encapsulation Process. Results may be seen in the examples shown for the composition in FIG. 8.

2.5.6 Assay Test No 6 Semi-Quantification of Aconitum Alkaloids

The extract obtained from the Encapsulation Process was pulverized into powder and passed through a 0.45 mm sieve. Five ml of hydrochloric acid (0.05 mol/L) was added to 0.4 g of the accurately weighted powder, and then sonicated for 30 minutes. The mixture was then extracted with ethyl acetate three times using 15 ml of ethyl acetate each time. The aqueous solution was then extracted again with chloroform three times, using 30 ml of chloroform for each extraction. The extracts recovered from the chloroform solvent were combined and evaporated to dryness under a stream of air to obtain a residue. The residue recovered was dissolved in 1 ml of hydrochloric acid (0.01 mol/L) and filtered through a 0.45 μm Millipore filter so as to make up the sample or test solution.

To prepare a reference solution, aconitine (the reference substance) was added to hydrochloric acid (0.01 mol/L) (1 ml:0.05 mg) and filtered through a 0.45 μm Millipore filter.

The High Performance Liquid Chromatography (HPLC) procedure below as performed according to the Chromatography Procedure on page 57 of the Appendices of Chinese Pharmacopoeia (Volume One, 2000 and edition). An ODS C₁₈ column was used as the stationary phase. Elution of the alkaloids was carried out using a mixture of acetonitrile and 10 mM ammonium bicarbonate buffer solution (adjusted with ammonia solution to pH 9.8±0.2), filtered through a 0.45 μm Millipore filter and degassed prior to use, in a gradient mode in which the ratio of acetonitrile at 0, 10, 20, 50, 75 and 80 min was 20%, 30%, 32%, 35%, 44% and 80%, respectively. The flow-rate was 1.0 mL/min. Detection was carried out at 240 nm with the reference wavelength of 550 nm at room temperature for 50 μL sample solution injected to the HPLC system.

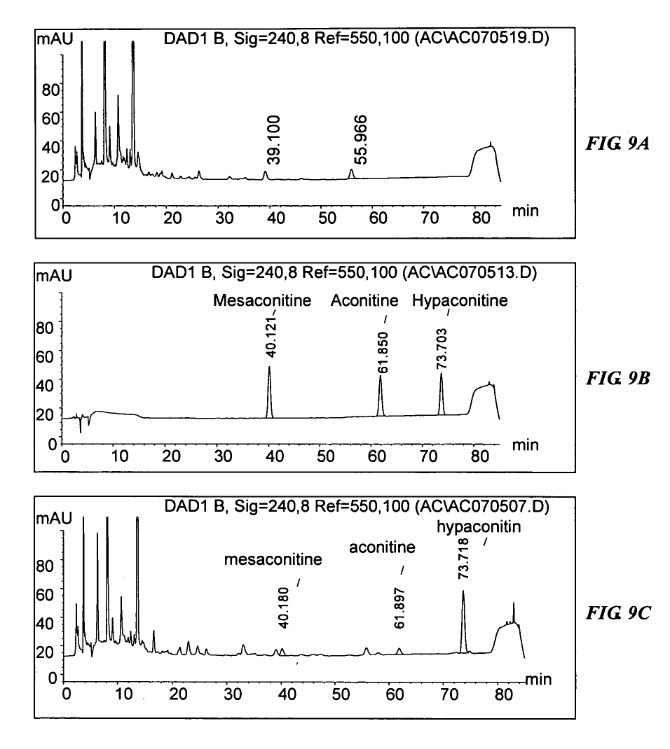

The peak area of aconitum alkaloids (including mesaconitine, hypaconitine and aconitine) contained in each 1.0 g of the composition (i.e., the usual amount in two capsule used for oral administration) should not be more than the peak area of 50 μg of aconitine (based on mesaconitine, hypaconitine and aconitine having the same absorbencies). Results may be seen in the examples shown for the composition in FIG. 9. FIG. 9A shows the HPLC chromatograms for the composition that prepared without Radix Aconiti Lateralis Preparata, while FIGS. 9B and 9C show the HPLC chromatograms for aconitine, mesaconitine, and hypaconitine references and aconitine, mesaconitine, and hypaconitine in the composition respectively.

2.5.7 Assay Test No 7, Quantification of Sinomenine, Paeoniflorin, Paeonol, Curcumin

The extract obtained from the Encapsulation Process was pulverized into powder and passed through a 0.45 mm sieve. Twenty ml of 50% ethyl alcohol was added to 0.15 g of the accurately weighted powder in a 25 mL volumetric flask, followed by sonication for 30 minutes. Next, the solution was diluted to 25 ml with 50% ethyl alcohol, and shaken well. The resultant solution was filtered through a 0.451 μm Millipore filter so as to make the sample or test solution.

To prepare a reference solution, add sinomenine, paeoniflorin, paeonol, curcumin (the reference substance) to 50% ethyl alcohol and filter through a 0.45 μm Millipore filter (1 ml:0.05 mg for sinomenine, 1 ml:0.24 mg for paeoniflorin, 1 ml:0.13 mg for paeonol, 1 ml:0.015 mg for curcumin).

The HPLC analysis was carried out according to the Chromatography Procedure on page 57 of the Appendices of Chinese Pharmacopoeia (Volume One, 2000 and edition). An ODS C₁₈ column was used as the stationary phase. Elution of the alkaloids was carried out using a mixture of acetonitrile and phosphate buffer solution (0.1% orthophosphoric acid with 0.2% triethylamine, pH 3.5±0.2), filtered through a 0.45 μm Millipore filter and degassed prior to use, in a gradient mode in which the ratio of acetonitrile at 0, 25, 30 and 55 min was 8%, 20%, 40% and 70%, respectively. The flow-rate was 1.0 mL/min. Detection was carried out at 240 nm for paeoniflorin, 262 nm for sinomenine, and 270 nm for paeonol, with the reference wavelength of 360 nm and 420 nm for curcumin. The reference wavelength of 550 nm at room temperature for the 5 μL sample solution injected to HPLC system.

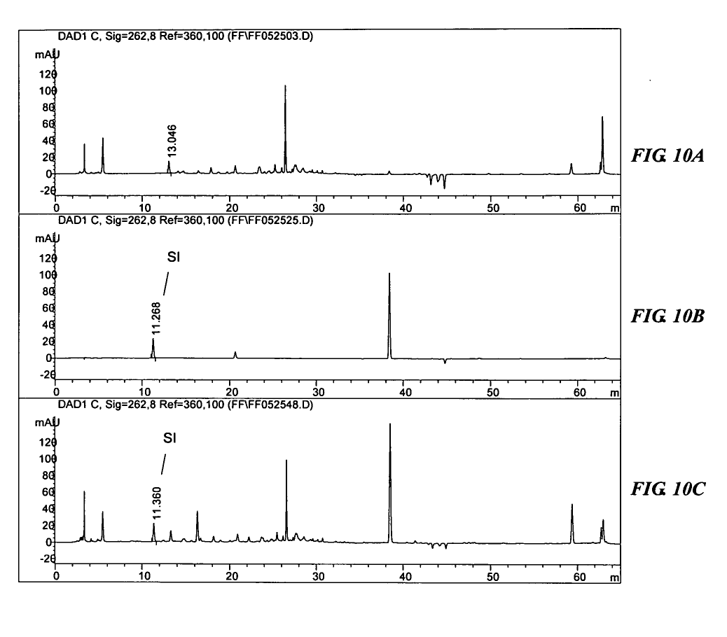

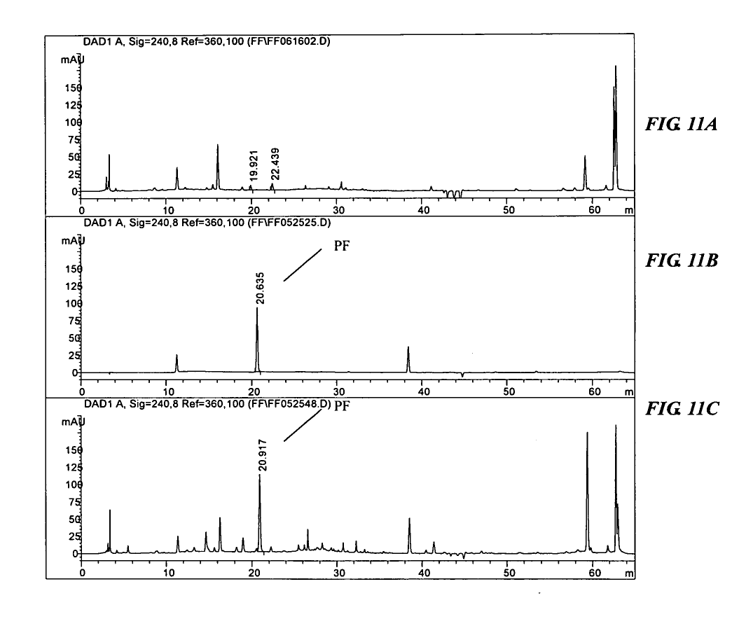

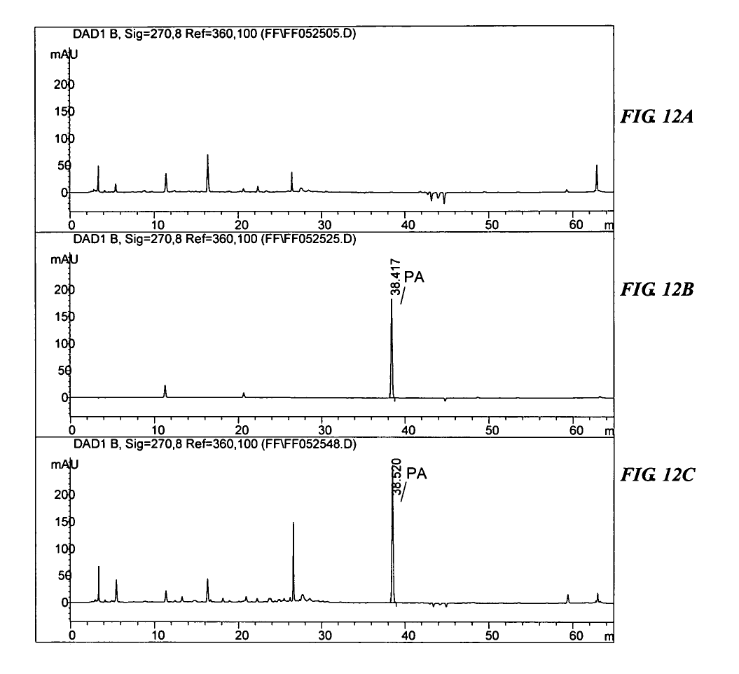

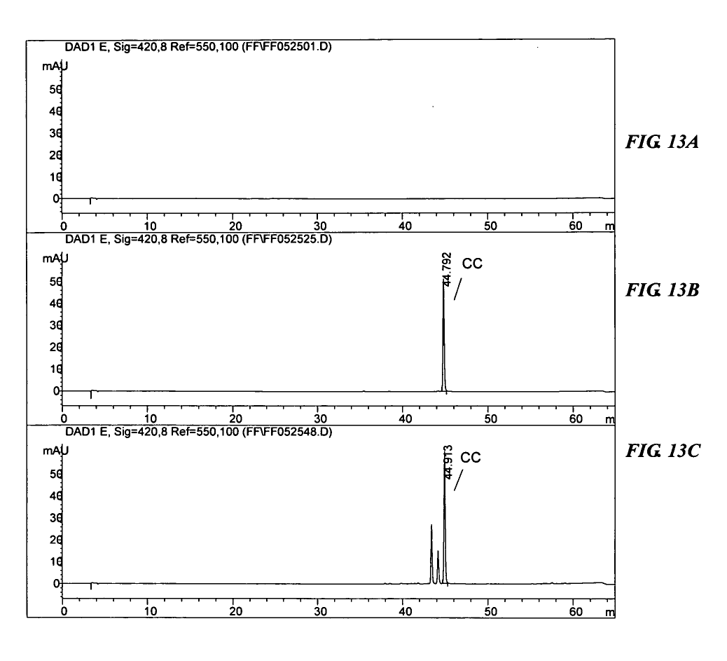

The amounts of sinomenine, paeoniflorin, paeonol, curcumin contained in each 1.0 g of the composition (i.e., the usual amount in two capsule used for oral administration) should not be less than 5.0 μg, 23.0 μg, 9.0 μg and 4.0 μg respectively. Results may be seen in the examples shown for the composition in FIGS. 10-13. FIG. 10A shows the HPLC chromatograms for the composition that prepared without Caulis Sinomenii, while FIGS. 10B and 10C show the HPLC chromatograms for sinomenine references and sinomenine in the composition respectively. FIG. 11A shows the HPLC chromatograms for the composition that prepared without Radix Paeoniae Alba, while FIGS. 11B and 11C show the HPLC chromatograms for paeoniflorin references and paeoniflorin in the composition respectively. FIG. 12A shows the HPLC chromatograms for the composition that prepared without Cortex Moutan, while FIGS. 12B and 12C show the HPLC chromatograms for paeonol references and paeonol in the composition respectively. FIG. 13A shows the HPLC chromatograms for the composition that prepared without Rhizoma Curcumae Longae, while FIGS. 13B and 13C show the HPLC chromatograms for curcumin references and curcumin in the composition respectively.

2.5.8 Assay Test No 8 Test Quantification of hypaconitine

The chromatography method and the sample solution prepared in Assay Test Number 6 (see above) was used for this assay.

To prepare a reference solution, add hypaconine (the reference substance) to hydrochloric acid (0.01 mol/L) (1 ml:0.010 mg) filter through a 0.45 μm Millipore filter.

The amounts of hypaconitine contained in each 1.0 g of the composition (i.e., the usual amount in two capsule used for oral administration) should not be less than 7 μg. Results may be seen in the examples shown for the composition in FIG. 9.

It may thus be seen that the above chromatographic analyses were used as quality control tests for the formulation of the present invention. The quality of the formulation may be asacertained or determined from the presence of prominent markers or major active ingredients. The qualitative or quantitative determination of the major active ingredient was done in comparison to known reference markers or standards of these active ingredients that were subjected concurrently to the chromatographic analyses.

A formulation that is incomplete or an extraction process that is unacceptable will result in the absence or poor yield of one or more of the markers or active ingredients analyzed. Such a formulation or extraction process will not result in a formulation that is of acceptable quality.

2.6 Animals

Rodents (rats and mice) were purchased from the Laboratory Animal Services Center of Chinese University of Hong Kong and the Laboratory Animal Services Center of Hong Kong University. Animals were kept in different cages (15 animals per cage for mice and 5 animals per cage for rats) and acclimatized for at least one week before experiment. They were fed standard laboratory rat chow and tap water ad libitum except for specific fasting periods before each experiment as indicated below. The animal laboratory was equipped with automatic temperature (22±1° C.) and lighting controls (12 hours light/12 hours dark).

All procedures involving animals and their care were approved by and carried out under the regulations of the Committee on the Use of Human & Animal Subjects in Teaching and Research (HASC) of Hong Kong Baptist University and Health Department of HKSAR and were conducted according to institutional guidelines that are in compliance with the United States' National Institutes of Health's Guide for the Care and Use of Laboratory Animals.

2.7 Drug administration

For administration to the animals, the herbal extracts were prepared in an emulsion form consisting of 10% of peanut oil, 10% of Tween-80 and the remaining 80% of the respective extracts, and was administered orally to animals. The dosage was expressed as herb weight/body weight. Control animals were administered the vehicle emulsion consisting of 10% of peanut oil, 10% of Tween-80 and 80% of water. For any dilution, the control emulsion or vehicle (Peanut oil: Tween®80: Water=1:1:8) was used. Positive control drugs were prepared with the vehicle emulsion.

2.8 Acute Inflammation Models

The anti-inflammatory effects of above extracts of the present invention were investigated in acute inflammatory models induced by either carrageenan or egg white.

Rats received a subplantar injection of carrageenan (0.1 ml of a 1% suspension in 0.85% saline; Sigma Chemical Co., St Louis, Mo., USA) or egg white (0.1 ml of a 10% solution in 0.85% saline) in the left hind paw. The total volume injected was 0.1 ml.

Animals fasted for 24 h before the experiment and the herbal extracts were given by gavage to animal 1 hour before, and 24 hours after the injection of carrageenan, or 3 hours before the injection of egg white at the dosages indicated in the respective tables in the results section. Drugs were administered in a staggered fashion to allow a consistent time to pass between each animal. Indomethacin was administered to animals in the positive control group.

Assessment of the Paw Edema Test

The volume of paw edema (in ml) was measured in each animal using a plethysmometer (Plethysmometer 7150, Ugo Basile, Italy) to a precision of two decimal places. The measurements were made immediately before and 2, 4, 6, 24 h after the carrageenan injection or 1, 2, 3, and 4 h after the injection of egg white. The swelling rate of paw was the percentage difference in paw volume and calculated by the following equation: Swelling rate (%)=(A−B)×100/B, where A represents the paw volume at different time points after injection, and B represents the paw volume before injection.

Values are presented as mean±SD. Analysis of variance (ANOVA) followed by post hoc analysis with LSD method (equal variance) or Tamhane's T2 method (unequal variance) were used for the evaluation of data and p<0.05 was accepted as statistically significant.

2.9 Antinociceptive Testing

Antinociceptive effects of the herbal extracts were used determined with the tail-flick test in rat and with the writhing test in mice.

2.9.1 Tail Flick Test

The tail-flick test is a model of acute pain. In this test, a radiant heat source is applied directly to the tail, and the latency is measured by the amount of time it takes for the animal to flick its tail aside. Spague-Dawley rats weighing 180 g to 220 g upon arrival were pre-selected prior to formal testing. These animals whose latency to respond was less than 2 s or more than 6 s were excluded from the experiments.

Animals were fasted for 24 h before the experiment and the herbal extracts were given 1 hr before the experiment at the dosages indicated in the respective tables in the results section. Drugs were administered in a staggered fashion to allow a consistent time to pass between each animal.

Each rat was then gently placed into the restrainer of the apparatus (IITC model 336 tail flick analgesia meter, IITC Inc., Woodland Hills, Calif., U.S.A) with the tail positioned in the tail groove. The tail-flick response was elicited by applying radiant heat to dorsal surface of the tail, ⅓ the length from the tip. The animal was observed until it showed a nociceptive response by flicking its tail aside or until the cut-off time (12 s to 15 s) was reached. The latency to respond was then recorded. For animals that did not respond prior to the cut-off time, the cut-off time was recorded. Adherence to the pre-selected cut-off time helped to minimize the tissue damage that could occur with prolonged exposure to radiant heat.

Assessment of the Tail-Flick Test

The tail-flick response was measured 1, 2, and 3 hours after the drug administration. Values are presented as mean±SD. Analysis of variance ANOVA followed by post hoc analysis. The LSD method (equal variance) or Tamhane's T2 method (unequal variance) was used for the evaluation of data and p<0.05 was accepted as statistically significant.

2.9.2 Writhing Test

In the writhing test, acetic acid is injected into the intraperitoneal cavity of mice. This induces acute inflammatory pain characterized by a contraction wave of abdominal musculature through stretching of the hind limbs that is called writhing, and the number of writhing episodes is evaluated. The writhing test possesses high sensitivity and makes it possible to detect analgesic activity of non-steroid anti-inflammatory drugs.

Male and female ICR mice weighing 18 g to 25 g were fasted for one day before the test. The herbal extracts were given 1 or 2 hr before the injection of acetic acid at the dosages indicated in the respective tables in the results section. The animals were then placed in individual 101×15 cm Plexiglas observation chambers for a 10-min acclimatization period. Acetic acid, 0.8% in 0.85% saline was injected i.p. into each mouse the animals were then returned into their respective transparent chambers.

2.9.3 Assessment of the Writhing Test

The animals were then observed for a specific period (15 or 20 mins) and the number of writhing episodes was counted. For scoring purpose, a writhe was indicated by a contraction wave of abdominal musculature with simultaneous stretching of at least one hind limb. The number of writhing episodes was measured 2 hr after drug administration, which was performed in a staggered fashion to allow a consistent time to pass among each animal. The dosages of the herbal extracts given are indicated in the respective tables in the results section.

Values are presented as mean±SD. Analysis of variance ANOVA followed by the post hoc analysis with LSD method (equal variance) or Tamhane's T2 method (unequal variance) were used for the evaluation of data and p<0.05 was accepted as statistically significant.

2.10 Experimental Arthritis

Adjuvant-induced arthritis (AA) and Collagen II induced arthritis (CIA) have been used extensively as models to study the immunological aspects of rheumatoid arthritis as well as to develop and test anti-rheumatoid arthritis drugs.

2.10.1 AA Using Mycobacterium tuberculosis

AA is the most classic and widely used animal model not only in delineating the pathological mechanism of inflammation and autoimmune phenomena, but also at the preclinical phase of the development process of potential antirheumatic drugs.

Preparation of Mycobacterium tuberculosis Suspension and Induction of AA

Heat-killed Mycobacterium tuberculosis H37Ra was obtained from Difco, Detroit, Mich., U.S.A; mineral oil was from Sigma-Aldrich Co., St. Louis, Mont., U.S.A; incomplete Freund's adjuvant 7001 (M. tuberculosis, 4.0 mg/ml, Lot 093003) was from Chondrex, LLC. Washington, U.S.A. Heat-killed M. tuberculosis was ground with incomplete Freund's adjuvant (IFA) in a glass mortar to obtain suspension of 0.625 mg/ml of M. tuberculosis. Male Sprague-Dawley rats weighing 100 g to 130 g at the start of the experiment were then injected subcutaneously at the tail base with 0.10 ml of the suspension to induce arthritis. Rats (n=7˜12 for each group) were treated with daily oral administrations of extracts starting right after immunization. Matched volumes of the emulsion or vehicle (Peanut oil:Tween-80:Water=1:1:8) were used as negative control.

Assessment of AA Using Mycobacterium tuberculosis

The development of AA was monitored by daily arthritic scoring as well as by the measurements of body weight and bi-hind paw volume every other day from day 9 onwards. Lesions of four paws were graded from 0 to 4 according to the extent of both erythema and edema of the periarticular tissue; a grade of 16 was the maximum arthritic score per animal. The hind paw volumes were measured using a plethysmometer chamber (Plethysmometer 7150, Ugo Basile, Italy). The volume of paw edema (ml) was measured in each animal to a precision of two decimal places. The data were expressed as the mean volume of both hind paws. The body weight of the rats was monitored with a 0.1 g precision balance (Sartorius, Germany).

Values are presented as mean±SD. Data were analyzed using the software package by Statistical Analysis System (SAS version 8.0). Repeated measures of a general linear model procedure were conducted at a significance level of alpha=0.05. The Student Newman-Keuls (SNK) post-hoc (alpha=0.05) test was used for a posteriori comparisons among individual groups.

2.10.2 Collagen-Induced Arthritis

Collagen-induced arthritis (CIA) is an experimental model of an autoimmune disease that can be elicited in susceptible strains of rodents (rat and mouse) by immunization with type II collagen. There are certain similarities with human condition including the linkage of disease to genes residing in histocompatibility locus, mononuclear cell infiltration, pannus development, fibrin deposition, erosion of cartilage and bone and autoreactive T and B cells. CIA has become industry standard by which potential therapeutic agents are evaluated. Following immunization, these animals develop an autoimmune-mediated polyarthritis that shares several clinical, histological, and immunological features with the human autoimmune disease rheumatoid arthritis. Because of the important similarities between CIA and rheumatoid arthritis, this experimental model has been the subject of extensive testing for the development of potential anti-inflammatory drugs.

Preparation of Type II Collagen and Induction of Arthritis

Bovine type II collagen in 0.05M acetic acid (Lot 082503) and Incomplete Freud's Adjuvant (Lot 091003) were purchased from Chondrex, LLC. Washington, U.S.A. The collagen and an equal volume of incomplete Freund's adjuvant (IFA) were processed with a homogenizer (IKA-WERKE Gmbh & Co. KG, Germany) for 30 sec in an ice water bath to form an emulsion. The ice water bath was crucial in preventing denaturation of the collagen during mixing as denatured collagen would not induce CIA.

Female Wistar rats weighing 200 g to 380 g at the start of the experiment were injected intradermally at two sites at the base of the tail with 0.10 ml of the type II collagen in Incomplete Freund's Adjuvant (CII/IFA) emulsion (0.05 ml at each site). A similar injection was administered on day 7 of the primary immunization as a booster. Rats (n=5-6 for each group) were treated by daily p.o. administration of extracts or indomethacin emulsion as a positive control from the day of induction of arthritis (day 0) until day 30. Negative control group of animals was administered by the emulsion or vehicle (Peanut oil:Tween-80:Water=1:1:8).

Assessment of Collagen Induced Arthritis

The development of collagen induced arthritis (CIA) was monitored by daily arthritic index scoring, and measurements of body weight and bi-hind paw volume taken every three days until day 30. Lesions of the four paws were graded from 0 to 4 according to the extent of both erythema and edema of the periarticular tissue with grade 16 as the maximum arthritic score per animal. The hind paw volume was measured using a plethysmometer chamber (U. Basile, Comerio, Italy), and expressed as the mean volume of both hind paws. The body weight of the rats was monitored with a 0.1 g precision balance (Sartorius, Germany).

Values are presented as mean±SD. Data was analyzed using the SAS software package (SAS version 8.0). Repeated measures of a general linear model procedure were conducted at a significance level of alpha=0.05. The Student Newman-Keuls (SNK) post-hoc (alpha=0.05) test was used for a posteriori comparisons among individual groups.

2.11 Toxicity

Toxicity studies were done to in two respects: examining the maximum tolerance dosage of Mixtures D, Dp and the capsule; and subchronic toxicity of the capsule.

2.11.1 Maximum Tolerance Dosage

In Mice

Male and female ICR mice weighing 18 g-26 g were fed standard laboratory chow and tap water ad libitum but were fasted 16 hr before the experiment. Two groups of mice were administered the Mixtures D and Dp by gavage in staggered fashion. The mortality, toxicity reaction and general condition of mice were observed between day 0 to day 6 and at day 13.

In Rats

Male and female SD rats weighing 110˜140 g were fed standard laboratory chow and tap water ad libitum but were fasted 16 hr before the experiment. Two groups of SD rat were administered the capsule and vehicle (0.3% carboxylmethyl cellulose sodium) by gavage in staggered fashion. The mortality, toxicity reaction and general condition of rats were observed between day 0 to day 13.

2.11.2 Subchronic Toxicity

Subchronic toxicity test is usually defined as studies of longer than 1 to 2 months duration, i.e., approximately 5% of the life span in the laboratory rat. Conducting subchronic toxicity tests may produce a toxic effect and to define a safety factor. And the subchronic study is designed to elucidate any of the myriad of potential toxic effects of a xenobiotic, i.e. Chinese medicine formula, on structural and functional entities.

Male and female SD rats weighing 90˜110 g were fed standard laboratory chow and tap water ad libitum. 3 groups of rats (3 rats for each group) were administrated by gavage in staggered fashion with the capsule at the dosages of 51.0 g/kg and 76.5 g/kg, or vehicle (0.3% 0.3% carboxylmethyl cellulose sodium). Animals were given drug once daily at 6 days per week. The mortality and toxicity reaction of the rats were observed every day. The administration volume was calculated according to the body weight at alternate day of each week.

3. Results

3.1 Determination of Optimal Process

The efficacies of the seven extracts by the different methods described above, i.e., processes A-G; were assessed by preliminary experiments to measure their anti-flammatory (egg white induced paw edema) and anti-nociceptive effects (Tables. 1-3), and on the extraction efficiencies of the selected main chemical components (Table 4).

3.1.1 Paw Edema Test

TABLE 1
Comparison on the anti-inflammation effect of extracts in paw edema test (N = 6)

| | | | Swelling percentage of paw volume after stimulation (%) | | | |
|---|---|---|---|---|---|---|
| Group | Tested sample | Dosage (g/kg) | 1 hr | 2 hr | 3 hr | 4 hr |
| A | nil | — | 45.50 ± 7.22 | 35.98 ± 6.74 | 24.17 ± 6.72 | 20.56 ± 8.90 |
| B | Dexamethasone | 8 mg/kg | 26.66 ± 3.34*** | 19.31 ± 5.83*** | 9.12 ± 4.42** | 7.49 ± 4.08 |
| C | Mixture A | 11.54 | 39.83 ± 7.96^{#} | 29.20 ± 7.87^{#} | 18.82 ± 5.40^{#} | 14.27 ± 6.43 |
| D | Mixture B | 11.54 | 40.31 ± 10.49^{#} | 27.04 ± 10.01^{#} | 16.84 ± 12.97 | 14.04 ± 11.69 |
| E | Mixture C | 11.54 | 40.93 ± 10.03^{##} | 24.47 ± 8.83* | 14.54 ± 6.85* | 10.82 ± 3.18 |
| F | Mixture D | 11.54 | 27.48 ± 7.61*** | 16.87 ± 7.54*** | 8.41 ± 8.27*** | 4.50 ± 5.80 |
| G | Mixture E | 11.54 | 33.86 ± 11.71* | 24.45 ± 12.66* | 18.56 ± 11.41^{#} | 10.02 ± 10.54 |
| H | Mixture F | 11.54 | 31.23 ± 5.30*** | 20.95 ± 2.04** | 14.76 ± 3.57* | 8.85 ± 1.90 |
| I | Mixture G | 11.54 | 29.81 ± 7.55*** | 16.95 ± 7.84*** | 13.82 ± 6.54* | 7.57 ± 6.49 |

Note
*P < 0.05,
**P < 0.01,
***P < 0.001, compared with the control group at same time point.
^{#}P < 0.05,
^{##}P < 0.01 compared with the Mixture D group at same time point.

3.1.2 Tail Flick Test

TABLE 2
Comparison on the antinociceptive effect of extracts in rats using the tail flick test

| | | | | Latency after drug administration(s) | | |
|---|---|---|---|---|---|---|
| Group | Tested sample | Animal No | Dosage (g/kg) | 60 min | 120 min | 180 min |
| A | nil | 7 | — | 7.09 ± 0.65 | 7.20 ± 0.97 | 7.22 ± 0.89 |
| B | Mixture A | 6 | 11.54 | 8.59 ± 0.69 | 7.61 ± 0.40 | 7.46 ± 0.94 |
| C | Mixture B | 6 | 11.54 | 9.49 ± 0.66** | 8.33 ± 1.18* | 7.48 ± 0.80 |
| D | Mixture C | 6 | 11.54 | 8.26 ± 1.40 | 7.93 ± 0.69 | 7.33 ± 1.05 |
| E | Mixture D | 5 | 11.54 | 9.09 ± 0.71* | 8.21 ± 0.80 | 6.47 ± 0.64 |
| F | Mixture E | 6 | 11.54 | 8.15 ± 1.10 | 7.98 ± 0.78 | 7.27 ± 0.98 |
| G | Mixture F | 5 | 11.54 | 7.04 ± 0.66^{#} | 6.86 ± 0.85 | 6.39 ± 0.79 |
| H | Mixture G | 5 | 11.54 | 7.98 ± 1.18 | 7.51 ± 1.56 | 6.69 ± 0.97 |

Note
*P < 0.05,
**P < 0.01,
***P < 0.001, compared with the control group at same time point.
^{#}P < 0.05,
^{##}P < 0.01 compared with the Mixture D group at same time point.

3.1.3 Writhing Test

TABLE 3
Comparison on the antinociceptive effect of
extracts in mice using the writhing test

| Group | Tested sample | Animal No. | Dosage (g/kg) | Count of writhe |
|---|---|---|---|---|
| A | Vehicle | 13 | N/A | 16.2 ± 9.5^{#} |
| B | Mixture A | 10 | 23.07 | 17.0 ± 11.3 |
| C | Mixture B | 13 | 23.07 | 6.0 ± 4.2 |
| D | Mixture C | 12 | 23.07 | 13.1 ± 3.7^{##} |
| E | Mixture D | 14 | 23.07 | 4.5 ± 3.5* |
| F | Mixture E | 15 | 23.07 | 6.6 ± 5.1 |
| G | Mixture F | 10 | 23.07 | 16.6 ± 4.4^{##} |
| H | Mixture G | 13 | 23.07 | 12.7 ± 6.9^{#} |

Note
*P < 0.05,
**P < 0.01,
***P < 0.001, compared with the control group at same time point.
^{#}P < 0.05,
^{##}P < 0.01 compared with the Mixture D group at same time point.

3.1.4 Extraction Efficiencies of the Various Processes

TABLE 4
Extraction efficiencies of main active components in various extraction methods (%, N = 2)

| | | | | | | | Total score for |
|---|---|---|---|---|---|---|---|
| Sample | Sinomenine | Paeoniflorin | Paeonol | Curcumin | demethoxycurcumin | Bisdemethoxycurcumin | extraction rate |
| Mixture A | 81.14 | 95.63 | 38.38 | 3.50 | 5.23 | 3.70 | 54.63 |
| Mixture B | 77.60 | 119.40 | 18.48 | 0.00 | 0.00 | 0.00 | 59.47 |
| Mixture C | 75.73 | 109.03 | 43.23 | 59.33 | 76.84 | 59.06 | 69.31 |
| Mixture D | 73.13 | 145.37 | 61.98 | 51.64 | 70.67 | 51.24 | 78.36 |
| Mixture E | 99.28 | 132.91 | 71.90 | 4.02 | 5.73 | 3.63 | 71.99 |
| Mixture F | 57.94 | 88.91 | 0.00 | 0.00 | 0.00 | 0.00 | 44.06 |
| Mixture G | 44.95 | 93.16 | 0.71 | 16.54 | 20.61 | 17.72 | 45.10 |

The experimental data (Table 1) showed that Mixture D had the most potent anti-inflammatory effects to the acute inflammation in rats evoked by injection of egg white. Mixture D also showed powerful anti-nociceptive effects to the acute pain reaction in mice induced by intraperitoneal acetic acid injection (Table 3). However, Mixture B exerted the most powerful anti-nociceptive effect to the acute pain reaction in rats induced by radiant stimulus using the tail-flick test (Table 2).

The extractions of desirable active compounds such as sinomenine, paeoniflorin, peonol, curcumin, demethoxycurcumin and bisdemethoxycurcumin were higher using Process D (Table 4) compared to the other extraction methods. As such, Mixture D, as obtained by Process D, was purified and used for subsequent experiments. While not as effective as Mixture D, the mixtures obtained by the other processes of the present invention also exhibit bioactive effects and remain under the scope of the present invention.

3.2 Comparison Between Mixtures D and Dp

Comparisons in the efficacies of Mixtures D and Dp were performed. The contents of sinomenine, paeoniflorin and curcumin in Mixtures D and Dp were quantified using high performance liquid chromatography (HPLC) and the recovery efficiencies of these three components were defined as and calculated as the ratio of the respective contents of the components in Mixtures D and Dp.

3.2.1 Comparison of the yield efficiencies of Mixture D and Mixture Dp

The weights of resultant extracts from Process D and Dp were measured and the yield efficiency for each process is defined as the ratio of the weights of the resultant extract and raw materials. The yield efficiencies of Extracts 1, 3 and 5 of Mixture D and Mixture Dp were 12.50% and 5.25%, respectively (Table 5). The purification efficiency was the ratio of difference of the yield rate of Mixture Dp and that of Mixture D against the yield rate of Mixture D. The purification efficiency denotes the relative amounts of substances removed by the purification process of Dp.

TABLE 5
Yield efficiencies of Mixture D and Mixture Dp (N = 2)

| | Weight of | Yield | Purification |
|---|---|---|---|
| | resultant extracts | efficiency | efficiency |
| Sample | (g/100 g herbs) | (%) | (%) |
| Extracts 1, 3 & 5 | 12.50 ± 0.50 | 12.50 ± 0.50 | N/A |
| of Mixture D | | | |
| Extracts 1, 3 & 5 | 5.15 ± 0.35 | 5.15 ± 0.35 | 58.80 |
| of Mixture Dp | | | |

3.2.2 Comparison on the Recovery Efficiencies of Bioactive Components

The contents of sinomenine, paeoniflorin and curcumin in the Extracts 1, 3 and 5 of Mixtures D and Dp were determined by HPLC. After purification, 85.11% of sinomenine, 99.11% of paeoniflorin, and 74.22% of curcumin in the Extracts 1, 3 and 5 of Mixture D were preserved in the corresponding part of Mixture Dp (Table 6).

TABLE 6
Recovery efficiencies of selected bioactive components

| | Sinomenine | | Paeoniflorin | | Curcumin | |
|---|---|---|---|---|---|---|
| | Amount (%) | Recovery | Amount (%) | Recovery | Amount (%) | Recovery |
| Sample | (n = 3) | efficiency (%) | (n = 3) | efficiency (%) | (n = 3) | efficiency (%) |
| Mixture D | 0.094 ± 0.001 | N/A | 0.408 ± 0.005 | N/A | 0.059 ± 0.001 | N/A |
| Mixture Dp | 0.153 ± 0.008 | 85.11 ± 2.25 | 0.774 ± 0.009 | 99.11 ± 0.21 | 0.084 ± 0.002 | 74.22 ± 6.89 |

3.2.3 Comparison of Effect on Acute Inflammation Evoked by Carrageenan Between Mixtures D and Dp

Positive control drug indomethacin inhibited the swelling within 7 hrs after drug administration but this action was not observed at 24 hrs after stimulation. On the other hand, Mixture D (15.36 g/kg) and Dp (0.96 g/kg and 15.36 g/kg) both exerted significant anti-inflammatory effect at 25 hrs after drug administration. At the lower dose of 0.96 g/kg, Mixture D showed significant effect only at 7 hrs and 25 hrs after drug administration. In contrast, the higher dose of Mixture D of 15.36 g/kg, and both the lower and higher doses of Mixture Dp demonstrated significant effect at 3 hrs, 5 hrs, 7 hrs and 25 hrs after drug administration (Table 7). Clear dose-response and time-response relationships were observed in Mixtures D and Dp at the concentrations and time points tested.

TABLE 7
Effect of Mixture D and Dp on rat paw edema evoked by carrageenan

| | | | | Percentage change in swelling after injecting carrageenan (%) | | | |
|---|---|---|---|---|---|---|---|
| Group | Dosage (g/kg) | | N | 2 hour | 4 hour | 6 hour | 24 hour |
| Control | Nil | | 10 | 18.55 ± 6.57 | 40.00 ± 14.10 | 61.37 ± 22.91 | 46.89 ± 20.60 |
| Indomethacin | 10 | mg/kg | 10 | 12.27 ± 4.61* | 15.12 ± 7.85*** | 15.32 ± 7.84*** | 35.36 ± 14.65 |
| Mixture D | 0.96 | g/kg | 10 | 14.36 ± 6.84 | 28.34 ± 18.15 | 37.66 ± 18.25* | 25.52 ± 10.79** |
| Mixture D | 15.36 | g/kg | 10 | 6.99 ± 4.42*** | 6.32 ± 5.58*** | 12.63 ± 12.92*** | 26.69 ± 13.15* |
| Mixture Dp | 0.96 | g/kg | 10 | 7.50 ± 4.58*** | 16.68 ± 13.67** | 31.55 ± 17.29** | 24.84 ± 14.26* |
| Mixture Dp | 15.36 | g/kg | 9 | 7.67 ± 5.48** | 4.02 ± 3.65*** | 10.36 ± 8.49*** | 23.62 ± 14.52* |

Note:
***P < 0.001;
**P < 0.01;
*P < 0.05, compared with the control group at same time point.

3.2.4 Comparison of the Anti-Inflammatory Potency of Mixture D and Dp by Egg White Induced Paw Edema Test in Rats

Mixtures D and Dp both exerted significant anti-inflammatory effects 4 hrs after drug administration by gavage. (Table 8) A clear time-response relationship was observed in Mixtures D and Dp at the time points tested.

TABLE 8
Effect of Mixture D and Dp on rat paw edema evoked by egg white

| | | | Percentage change in swelling after injecting egg white (%) | | | |
|---|---|---|---|---|---|---|
| Group | Dosage (g/kg) | N | 1 hour | 2 hour | 3 hour | 4 hour |
| Control | nil | 10 | 41.40 ± 7.074 | 28.56 ± 5.305 | 24.64 ± 4.556 | 18.14 ± 2.987 |
| Mixture D | 0.96 | 10 | 32.44 ± 4.921* | 23.72 ± 4.954* | 16.98 ± 4.341* | 14.63 ± 3.877*,^{#} |
| Mixture Dp | 0.96 | 10 | 27.40 ± 5.431*** | 15.48 ± 4.336*** | 10.57 ± 5.630*** | 5.05 ± 4.554*** |

Note:
***P < 0.001;
**P < 0.01;
*P < 0.05, compared with the control group at same time point;
^{#}P < 0.05, compared with the group of Mixture Dp at same time point.

3.2.5 Comparison on the Anti-Nociceptive Potency of Mixture D and Dp in Rat Tail Flick Reaction Induced by Radiant Stimulus

Mixtures D and Dp both significantly prolonged the latency of rats to radiant stimulus at 1 hr after drug administration by gavage. Mixture Dp also significantly prolonged the latency at 2 hrs after drug administration (Table 9). A clear time-response relationship was observed in Mixture D and Dp at the time points tested.

TABLE 9
Effect of Mixture D and Dp on the latency of rat to radiant heat stimulation

| | | | Latency (s) | | |
|---|---|---|---|---|---|
| Group | Dosage (g/kg) | N | 60 min | 120 min | 180 min |
| Control | | 10 | 7.67 ± 1.32 | 8.02 ± 0.95 | 8.62 ± 1.44 |
| Mixture D | 7.69 | 9 | 10.30 ± 1.21** | 9.04 ± 1.18 | 8.58 ± 1.03 |
| Mixture Dp | 7.69 | 10 | 10.12 ± 1.08** | 9.43 ± 1.19** | 8.59 ± 1.28 |

Note:
*P < 0.05,
**P < 0.01 compared with the control group at same time point.

3.2.6 Writhing Test Induced by Acetic Acid Injection

Two hrs after administration, Mixture D and Dp both had similar significant inhibition to the writhing activity of mice induced by acetic acid. (Table 10)

TABLE 10
Effect of Mixture D and Dp on the
number of writhing episode in mice

| | | | The number of |
|---|---|---|---|
| Group | Dosage (g/kg) | N | writhing episodes |
| Control | N/A | 11 | 25.9 ± 15.8 |
| Mixture D | 23.07 | 10 | 11.8 ± 8.0** |
| Mixture Dp | 23.07 | 10 | 12.3 ± 7.4** |

Note:
*P < 0.05,
**P < 0.01 compared with the control group.

3.2.7 Acute Toxicity
Mortality

Mortality was calculated as number of animal deaths/number of animal used * 100. The mortalities of the mice given Mixture D and Dp were 65.2% and 10.5%, respectively (Table 11). The first case of death happened at 0.62 hr and 0.46 hr respectively, after the drug administration of Mixture D and Dp.

TABLE 11
Mortality of ICR mice after receiving Mixture D or Mixture Dp

| | Dosage | Animal | Dead | Mortality |
|---|---|---|---|---|
| Group | (g herb)/kg | No. | animal | (%) |
| Mixture D | 153.8 | 66 | 43 | 65.2 |
| Mixture Dp | 153.8 | 38 | 4 | 10.5 |

Body Weight

The body weight of the surviving ICR mice kept increasing after the administration of Mixtures D and Dp throughout the 13 days of the study. At day 13, the percentage of weight gain for Mixture D and Dp were 19.47% and 32.30% respectively (Table 12).

TABLE 12
Body weight gain of ICR mice after
receiving Mixture D or Mixture Dp

| | Mixture D (N = 23) | | Mixture Dp (N = 34) | |
|---|---|---|---|---|
| | Body | Body weight | Body | Body weight |
| Time | weight (g) | gain (%) | weight (g) | gain (%) |
| Day 0 | 22.63 ± 3.800 | 0 | 21.65 ± 3.198 | 0 |
| Day 1 | 21.98 ± 4.573 | −2.86 | 23.59 ± 3.470 | 8.98 |
| Day 2 | 23.53 ± 3.936 | −0.45 | 24.56 ± 3.484 | 13.44 |
| Day 3 | 23.57 ± 3.808 | 4.17 | 25.46 ± 3.232 | 17.6 |
| Day 4 | 23.83 ± 3.708 | 5.28 | 25.65 ± 3.283 | 18.51 |
| Day 5 | 24.30 ± 3.501 | 7.4 | 25.95 ± 2.837 | 19.86 |
| Day 6 | 24.57 ± 3.385 | 8.57 | 25.92 ± 2.804 | 19.72 |
| Day 13 | 27.04 ± 3.498 | 19.47 | 28.64 ± 2.804 | 32.3 |

Acute Toxicity: General Condition

Mortality in mice after drug administration was preceded by coma in all cases and was accompanied by clonic and tonic convulsions. In addition, sedation, labored breathing and swollen noses stained with porphyria were also seen before death.

For the surviving mice, 13.0% and 60.9% of overall mice after Mixture D administration showed fair condition in stool and fur respectively. Also, 38.9% and 11.1% of overall mice after Mixture Dp administration showed fair condition in stool and fur. These conditions were maintained for 1-2 days.

3.3 Bioactivities of Mixture Dp

3.3.1 Mixture Dp in Experimental Arthritis

Collagen-Induced Arthritis-(CIA)

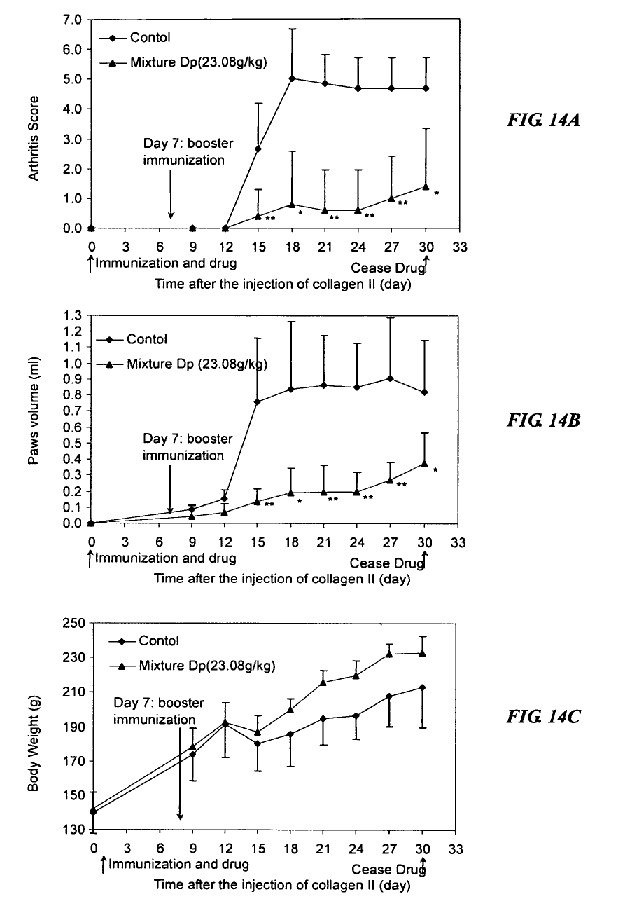

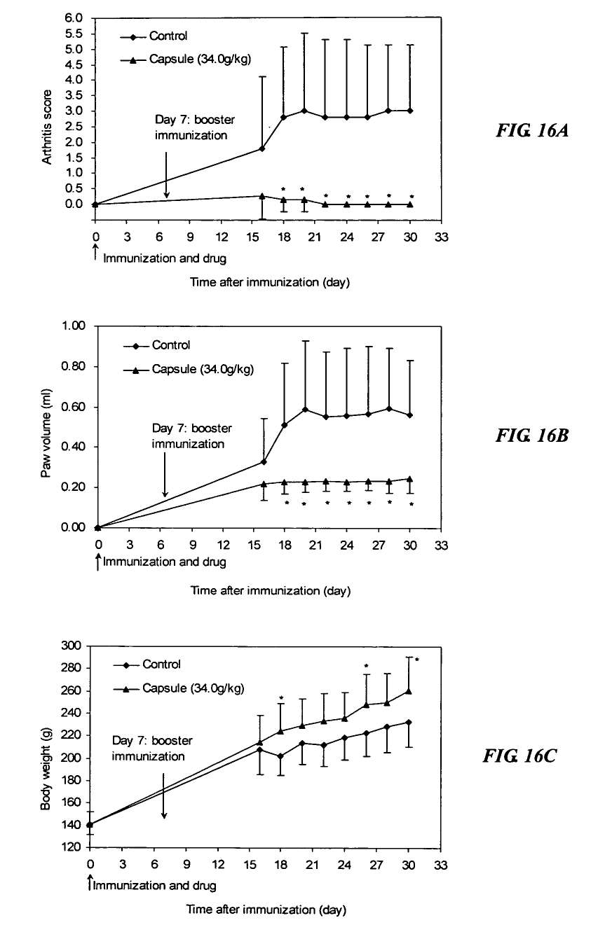

Following the immunization of CII/IFA, all animals in the negative control group began to develop arthritis from day 13 to day 16 onwards (FIG. 14). The arthritis score peaked on day 18, varied a little from day 19 to day 23 (FIG. 14A), and then remained stable to the end of the experiment, while the hind paw volume reached a plateau as late as day 27 (FIG. 14B). The diseased animals displayed marked loss of body weights (FIG. 14C). In contrast, daily p.o. administration of 23.08 g herbs/kg of Mixture Dp significantly inhibited the development of arthritis (FIGS. 16A and B). Mixture Dp significantly inhibited the increases in paw volume induced by collagen II (FIG. 14B) and reversed the trend in arthritic scores (FIG. 14A) in comparison with the control group. Mixture Dp also had influence on the body weight of CIA rats (FIG. 14C).

In FIG. 14, rats were treated p.o. daily with Mixture Dp (▴, n=5) from the day of the induction of CII/IFA (day 0) until day 30. The effects of the capsule on the arthritic score (A), hind paw volume (B), and body weight (C), were compared with those of treatment of CIA with vehicle (♦, n=10). Significant inhibitions of the development of CIA in arthritis scores, hind paw volume, and body weight were observed. Data were expressed as means±SD; * P<0.05 compared with the vehicle-treated CIA rats.

3.3.2 Mixture Dp in Acute Inflammation Models Inhibitory Effect of Mixture Dp on the Rat Model of Paw Edema Evoked by Carrageenan

The subplantar injection of carrageenan induced an obvious swelling lasting at least 24 hrs in the paw of rat where injection being performed, and the peak time were 6 hours after the injection. Positive control drug indomethacin inhibited the swelling within 7 hrs after drug administration by gavage, this action wasn't observed at 24 hrs after stimulation. Mixture Dp at 0.96 g/kg and 15.38 g/kg exerted a significant anti-inflammatory effect in 25 hrs after drug administration by gavage. A clear dose-response and time-response relationships were observed with tested concentrations and time points. (Table 13)

TABLE 13
Effect of Mixture Dp on the rat model of paw edema evoked by carrageenan

| | | | Percentage change in swelling after injection of carrageenan (%) | | | |
|---|---|---|---|---|---|---|
| Group | Dosage (g/kg) | N | 2 hour | 4 hour | 6 hour | 24 hour |
| Control | | 10 | 18.55 ± 6.57 | 40.00 ± 14.10 | 61.37 ± 22.91 | 46.89 ± 20.60 |
| Indomethacin | 10 mg/kg | 10 | 12.27 ± 4.61* | 15.12 ± 7.85*** | 15.32 ± 7.84*** | 35.36 ± 14.65 |
| Mixture Dp (Low) | 0.96 | 10 | 7.50 ± 4.58*** | 16.68 ± 13.67** | 31.55 ± 17.29** | 24.84 ± 14.26* |
| Mixture Dp (High) | 15.38 | 9 | 7.67 ± 5.48** | 4.02 ± 3.65*** | 10.36 ± 8.49*** | 23.62 ± 14.52* |

Note:
***P < 0.001;
**P < 0.01;
*P < 0.05, compared with the control group at same time point

Inhibitory Effects of Mixture Dp the Rat Model of Paw Edema Evoked by Egg White

The subplantar injection of egg white induced an obvious swelling lasting about 4 hrs in the paw of rat where injection being performed, and the peak time was 1 hour after the injection.

Mixture Dp at 0.24 g/kg, 0.96 g/kg and 15.36 g/kg exerted significant anti-inflammatory effect in 6 hrs after drug administration by gavage. But at 3.85 g/kg, Mixture Dp didn't show a significant anti-inflammatory effect, although a tendency of the swelling to decrease was observed. A clear time-response relationship was observed in Dp with tested time points. (Table 14)

TABLE 14
Effect of Mixture Dp on the rat model of paw edema evoked by egg white

| | | | Percentage change in swelling after injection of egg white (%) | | | |
|---|---|---|---|---|---|---|
| Group | Dosage (g/kg) | N | 1 hour | 2 hour | 3 hour | 4 hour |
| Control | | 9 | 45.77 ± 9.21 | 28.76 ± 6.51 | 23.98 ± 6.69 | 17.08 ± 5.37 |
| Mixture Dp (Low) | 0.24 | 10 | 32.54 ± 9.89** | 20.69 ± 8.67* | 13.93 ± 8.74* | 11.64 ± 7.49 |
| Mixture Dp (Medium 1) | 0.96 | 10 | 32.23 ± 6.79** | 21.60 ± 6.47* | 15.24 ± 4.86** | 10.25 ± 3.80** |
| Mixture Dp (Medium 2) | 3.85 | 8 | 38.47 ± 10.16 | 24.04 ± 7.41 | 18.16 ± 5.32 | 14.08 ± 6.21 |
| Mixture Dp (High) | 15.38 | 10 | 30.99 ± 5.12*** | 19.52 ± 6.29** | 14.81 ± 6.30** | 10.28 ± 7.06* |

Note:
***P < 0.001;
**P < 0.01;
*P < 0.05, compared with the control group at same time point

3.3.3 Anti-Nociceptive Effects of Mixture Dp
Tail-Flick Test

Mixture Dp at 7.69 and 30.77 g/kg significantly prolonged the latency of rats to radiant stimulation 1 hr after drug administration by gavage. Furthermore, Mixture Dp at 30.77 g/kg also significantly prolonged the latency to radiant heat 2 and 3 hrs after drug administration. But at 1.92 g/kg, Mixture Dp didn't show a significant anti-nociceptive effect, although a tendency of increasing latency was observed. A clear time-response relationship was observed in Mixture Dp at the time points tested. (Table 15).

TABLE 15
Effect of Mixture Dp on the latency of rats to radiant heat stimulation

| | | | Latency (s) | | |
|---|---|---|---|---|---|
| Group | N | Dosage (g/kg) | 60 min | 120 min | 180 min |
| Control | 10 | | 8.73 ± 1.79 | 8.80 ± 1.80 | 9.36 ± 1.38 |
| Mixture Dp (Low) | 8 | 1.92 | 9.34 ± 2.03 | 9.13 ± 1.25 | 9.17 ± 1.49 |
| Mixture Dp (Medium) | 9 | 7.69 | 10.64 ± 1.88* | 9.67 ± 1.41 | 9.01 ± 1.26 |
| Mixture Dp (High) | 8 | 30.77 | 18.62 ± 2.50** | 15.45 ± 3.01** | 11.44 ± 2.20** |

Note:
*P < 0.05,
**P < 0.01 compared with the control group at same time point

Writhing Test

Two hours after administration of Mixture Dp at 15.38 g/kg and 30.77 g/kg, both dosages caused a significant inhibition to the writhing activity of mice induced by acetic acid. A clear dose-response relationships was observed in Mixture Dp at the dosages tested. (Table 16)

TABLE 16
Effect of Mixture D and Dp on the
number of writhing episode of mice

| | | | The number of |
|---|---|---|---|
| Group | N | Dosage (g/kg) | writhing episodes |
| Control | 14 | | 14.5 ± 9.9 |
| Mixture Dp (Low) | 12 | 7.69 | 13.2 ± 17.0 |
| Mixture Dp (Medium) | 12 | 15.38 | 4.7 ± 4.0* |
| Mixture Dp (High) | 12 | 30.77 | 1.7 ± 2.0** |

Note:
*P < 0.05,
**P < 0.01 compared with the control group.

3.4 Bioactivities of the Capsule Produced under the Second Implementation
3.4.1 The Capsule in Experimental Arthritis
AA Using Mycobacterium tuberculosis

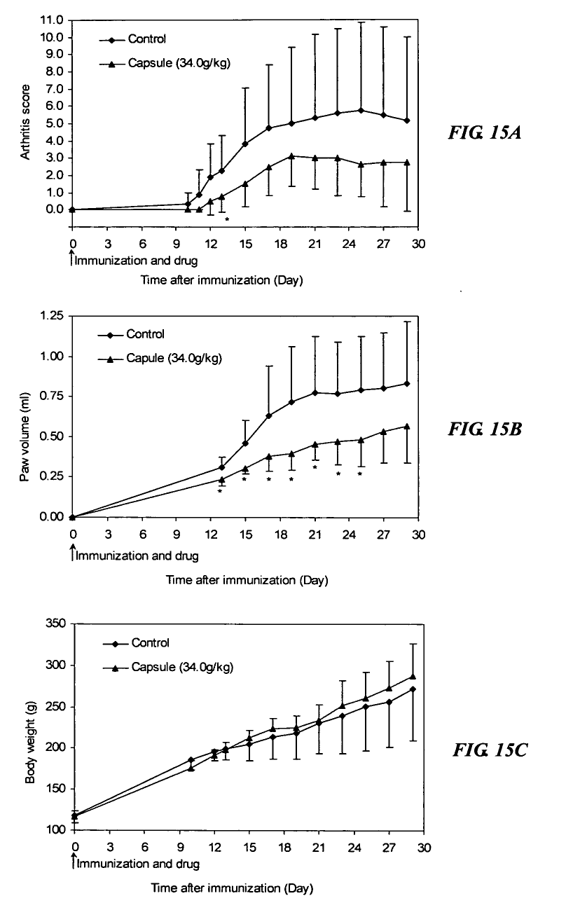

Following the immunization by adjuvant, animals began to develop arthritis from day 12 to day 21 (FIG. 15). The capsule significantly inhibited the increases in paw volume induced by adjuvant arthritis (FIG. 15B) and reversed the trend in arthritic scores (FIG. 15A) in comparison with the control group. The capsule had no marked influence on the body weight of AA rats (FIG. 15C).

In FIG. 15, rats were treated p.o. daily with the capsule (▴, n=7) from the day of the induction of CFA (day 0) until day 29. The effects of the capsule on the arthritic score (A), hind paw volume (B), and body weight (C), were compared with those of treatment of CIA with vehicle (♦, n=12). Significant inhibitions of the development of AA in arthritis scores, as well as hind paw volume were observed. Data were expressed as means±SD; * P<0.05 compared with the vehicle-treated AA rats.

Collagen-Induced Arthritis (CIA)

Following the immunization of CII/IFA, all animals in the negative control group began to develop arthritis from day 13 to day 16 onwards (FIG. 16). The arthritis score peaked on day 18, varied a little from day 19 to day 23 (FIG. 16A), and then remained stable to the end of the experiment, while the hind paw volume reached a plateau as late as day 27 (FIG. 16B). The diseased animals displayed marked loss of body weights (FIG. 16C). In contrast, daily p.o. administration of 34.0 g herbs/kg of the capsule significantly inhibited the development of arthritis (FIGS. 16A, B and C). The capsule significantly inhibited the increases in paw volume induced by collagen II (FIG. 16B) and reversed the trend in arthritic scores (FIG. 16A) in comparison with the control group. The capsule also had significant influence on the body weight of CIA rats (FIG. 16C).

In FIG. 16, rats were treated p.o. daily with the capsule (▴, n=5) from the day of the induction of CII/IFA (day 0) until day 30. The effects of the capsule on the arthritic score (A), hind paw volume (B), and body weight (C), were compared with those of treatment of CIA with vehicle (♦, n=10). Significant inhibitions of the development of CIA in arthritis scores, hind paw volume, and body weight were observed. Data were expressed as means±SD; * P<0.05 compared with the vehicle-treated CIA rats.

3.4.2 Anti-Nociceptive Effects of the Capsule

Writhing Test

One hour after administration of positive control tetrahydropalmatine at 0.09 g/kg and sinomenine at 0.0375 g/kg, and the capsule at 6.375 g/kg, 12.750 g/kg and 25.50 g/kg, all tested drugs caused inhibition to the writhing activity of mice induced by acetic acid. The inhibition by tetrahydropalmatine, sinomenine and 12.7 g/kg and 25.50 g/kg of the capsule was significant compared to control group. A clear dose-response relationship was observed in the capsule at the dosages tested. (Table 17)

TABLE 17
Effect of the capsule on the number of writhing episode of mice

| | | | The number of |
|---|---|---|---|
| Group | Dosage (g/kg) | N | writhing episodes |
| Control | Nil | 10 | 38 ± 10 |
| Tetrahydropalmatine | 0.090 | 10 | 11 ± 6** |
| Sinomenine | 0.0375 | 10 | 25 ± 9** |
| Capsule (Low) | 6.375 | 10 | 29 ± 9 |
| Capsule (Medium) | 12.750 | 10 | 29 ± 8* |
| Capsule (High) | 25.500 | 10 | 22 ± 11** |

Note:
*P < 0.05,
**P < 0.01 compared with the control group.

3.4.3 Toxicity of the Capsule
Acute Toxicity

Mortality wasn't found in 14 days after the single drug administration of the capsule at the dosage of 294 g herb/kg. In comparison with control group, at days 4 and 7, there was slight reduction in body weight gain (Table 18). Similarly, at days 2 and 5, there was slight loss of appetite (Table 19). One week after drug administration, these indexes recovered gradually.

TABLE 18
Body weight gain of SD rats after receiving the capsule (N = 10)

| | Body weight gain (g) | | | | |
|---|---|---|---|---|---|
| Group | Day 0 | Day 4 | Day 7 | Day 10 | Day 13 |
| Control | 131.23 ± 5.80 | 171.56 ± 10.19 | 197.92 ± 17.37 | 217.74 ± 25.31 | 232.35 ± 29.65 |
| Capsule | 134.77 ± 4.94 | 158.23 ± 8.24* | 185.40 ± 11.41* | 207.83 ± 16.69 | 228.18 ± 22.78 |

TABLE 19
Appetite of SD rats after receiving the capsule (N = 10)

| | Appetite (g) | | | |
|---|---|---|---|---|
| Group | Day 2 | Day 6 | Day 8 | Day 12 |
| Control | 24.50 ± 0.11 | 11.55 ± 8.38 | 23.90 ± 2.32 | 26.75 ± 6.48 |
| Capsule | 5.75 ± 1.47* | 4.70 ± 1.98* | 25.03 ± 1.52 | 23.73 ± 4.02 |

Subchronic Toxicity

Mortality was calculated as number of animal death/number of animal used *100%. No mortality was found up to 97 days after drug administration at the dosage of 51.0 and 76.5 g herb/kg.

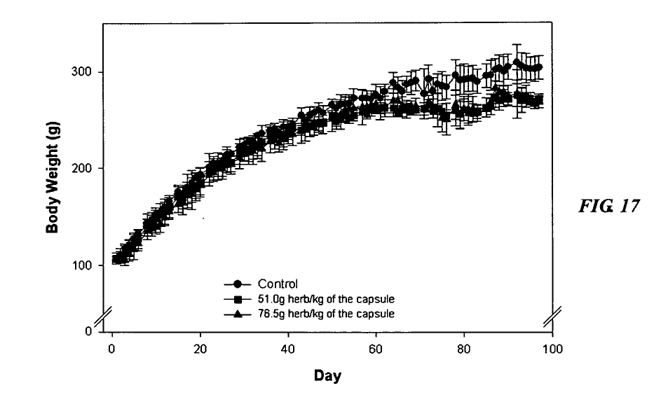

The body weight of animals in each group was kept increasing after the drug throughout 97 days of the experiment. At day 97, the percentage for weight gain for rats after administration of 0.3% CMC-Na solution, 51.0 g herb/kg and 76.5 g herb/kg of the capsule were 183.7%, 151.9% and 154.8% respectively (FIG. 17).

General Condition

The general condition observation mainly included any changes in the skin, fur, eyes mucus membrane, circulatory system, autonomic and central nervous systems, somatomotor activities, behavior. Any pharmacotoxic signs such as tremor, convulsions, salivation, diarrhea, lethargy, sleepiness, morbidity, fasciculation, mydriasis, miosis, droppings, discharges, or hypotonia was recorded.

No abnormal observation was found in rats given with 0.3% CMC-Na solution. Piloerection resulting in rough hair was found in rats given with 51.0 g herb/kg of the capsule from day 20 to day 97 and wet stool with slight severity was also found from day 30 onward. For rats administrated with 76.5 g herb/kg of the capsule, piloerection and wet stool with slight severity were both found from day 9 to day 97.

4. Discussion

4.1 Comparison of the Seven Processes

The comparison of seven processes in bioactivities and chemicals extraction aimed to screen and optimize the extraction method of component herbal of the formulation. The result showed that Mixture D which produced from Process D was the best in terms of the bioactivities, including acute anti-inflammatory effect in carrageenan induced paw edema, analgesia in rat tail flick reaction induced by radiant stimulation and mouse writhing reaction induced by acetic acid stimulation in peritoneal membrane and extraction efficiency to main active chemical component, including sinomenine, paeoniflorin, paeonol and curcumin, demethoxycurcumin and bisdemethoxycurcumin.

As such, Mixture D, as obtained by Process D, was purified and used for subsequent experiments. Nevertheless, the mixtures obtained by the other processes have utility and remain under the scope of the present invention.

4.2 Purification of Mixture Dp

The purification process aimed to remove biologically inactive substances from the extracts, thereby increasing the abundance of the active substances.

Polymeric adsorption resins are ideal materials to remove inactive substances from herbal extracts. These resins are being employed more and more in the purification processes of Chinese medicines due to their common selective adsorption property to glycosides, alkaloids, flavones, and terpenoids—substances that have potent biological activities and therefore are considered as active components in Chinese medicine. The Amberlite XAD-7HP resin is an ideal polymeric adsorption resin with medium polarity and it can selectively adsorb compounds with medium polarities such as sinomenine, paeoniflorin, and others of the present invention.

Our results (Table 5) showed that the purification efficiency is about 60% in which approximately 60% of substances in Mixture D have been removed by the Amberlite XAD-7HP resin (definition of the term “purification efficiency” given in Section 3.2.1 above). While the use of Amberlite XAD-7HP resin was taught, the invention is not limited to the use of this resin for the further purification of the herbal mixtures of the present invention.

Further chemical comparison on the recovery efficiencies of major chemical components (Table 6) and the main pharmacological activities and acute toxicity of the original extract (Mixture D) and resultant extract (Mixture Dp) (Table 7-10) demonstrated that these two extracts are almost pharmacologically equivalent and chemically similar, meanwhile, the acute toxicity of Mixture Dp was decreased (Table 11).

For the recovery efficiencies, the results indicated that 85.11% of sinomenine, 99.11% of paeoniflorin, and 74.22% curcumin were recovered from the purification process with Amberlite XAD-7HP resin (Table 6). According to the standard recommended by the Chinese State Food and Drug Administration's (SFDA), the recovery efficiency in each pharmaceutical process should not be less than 70%. Our results showed that the recovery efficiencies of the major components of the present invention were higher than 70% and therefore complies with this standard of the SFDA. This also implied that most of the active components are largely persevered through the purification process. This is very important maintaining the original pharmacological and clinical effects of the formula.

For the anti-inflammatory activities, the results Tables 7 and 8 showed that Mixture D and Dp have similar effects. In some case, Mixture Dp was superior to Mixture D. This may be seen at the lower dose of 0.96 g/kg at 3 and 5 hours after drug administration in carrageenan induced paw edema test (Table 7), and at 7 hours after drug administration in egg white induced paw edema test (Table 8). For anti-nociceptive activities, the results demonstrated that Mixture D and Dp also had close effects both in the rat tail flick test and mice writhing test (Tables 9 and 10). Therefore, the purified Mixture Dp has similar potency in anti-inflammatory effect as well as in anti-nociceptive as that of Mixture D.

The mortalities of animal after receiving Mixture D and Dp were 65.2% and 10.5% respectively (Table 11). This implies that the potentially toxic components of Mixture D were removed after passage through the XAD-7 column. The body weight of the surviving mice kept increasing through the duration of the study. This implied that both Mixture D and Dp do not have a suppressive influence on growth. The fair condition of stools implied that Mixture D and Dp may cause some irritation in the gastrointestinal tract of the animals. These results indicate that Mixture Dp could be less toxic than Mixture D in an acute toxicity test.

Thus, the purified fraction and Mixture D are basically similar both chemically and pharmacologically, while the purification procedure decreased the acute toxicity of Mixture D. The use of Amberlite XAD-7HP resin in the purification process can remove inactive components, preserve pharmacological activities, at the same time lower down the toxicity of Mixture D, therefore is acceptable.

4.3 The Bioactivities of Mixture Dp

The results showed that Mixture Dp of the present invention had significant anti-arthritic effects on the collagen II induced (FIG. 14) arthritis and anti-inflammatory effects on the paw edema either evoked by carrageenan (Table 13) or by egg white (Table 14) as well as an anti-nociceptive effect on the pain reaction induced by radiant stimulus on rats (Table 15) or by acetic acid on mice (Table 16). In paw edema tests, Mixture Dp exerted a significant anti-inflammatory effect at the dose of 0.96 g/kg.

But in anti-nociceptive activities, Mixture Dp could only inhibit the pain reaction of animal with the minimum doses of 7.69 g/kg (tail-flick test) and at 15.38 g/kg (writhing test). This suggests that Mixture Dp would have a more potent anti-inflammatory activity than on anti-nociceptive activity, which could be in favor of the treatment of rheumatoid arthritis by focusing on the pathogenesis of the disease. In the present study, the potent anti-arthritic effect of Mixture Dp was primarily shown and defined.

4.4 The Bioactivities of the Capsule

The results showed that the capsule of the present invention had significant anti-arthritic effects on the adjuvant induced arthritis and collagen II induced arthritis (FIGS. 15 and 16) as well as an anti-nociceptive effect on the pain reaction induced by acetic acid on mice (Table 17).

4.5 The Toxicity of the Capsule

The results showed that the capsule of the present invention was low in acute (Tables 18 and 19) and subchronic toxicity (FIG. 17). When giving orally 294 g/kg of the capsule once, no mortality was found in SD rats. The major toxic signs were loss of body weight gain and loss of appetite in the first week of drug administration, which recovered in the second week. When giving orally 51.0 or 76.5 g/kg of the capsule 6 days a week, total 97 days, similar results were observed.

4.6 Chemical Composition and Proportions

While these main chemical components (sinomenine, paeoniflorin, peonol, curcumin, demethoxycurcumin and bisdemethoxycurcumin) and their effects are individually known in the art, their use as exemplified by extraction from the combination of herbs of the present invention is not obvious even to one of skill in the art. The extracts of the present invention may comprise many other components that have not been identified or quantified. Nevertheless, the novel combination of the known main active ingredients of sinomenine, paeoniflorin, peonol, curcumin, demethoxycurcumin and bisdemethoxycurcumin in a formulation has hitherto not been taught in the prior art. As such, an oral composition comprising these main ingredients as identified from the present invention, in essentially or substantially purified form from the named list of herbs above, or in artificially synthesized form, come under the scope of the present invention

Also, while the above examples teach how the present invention may be implemented, it is clear that the proportion or ratio of the components is more important than the unit of mass used. Thus, a person skilled in the art can scale up or down when practicing the invention while keeping to the proportions or ratios taught, and remain within the scope of the invention. Where units of mass are specified with regards to the formulation, it may be understood that the invention is not limited by the unit of mass used.

4.7 Other Oral Formulations of the Present Invention Suitable for Human Use

Besides capsules, the extracts may be provided as a tablet, aqueous or oil suspension, dispersible powder or granule, emulsion, hard or soft capsule, syrup, elixir, or beverage. Formulations intended for oral use may be prepared according to any method known in the art for the manufacture of pharmaceutically acceptable formulations and such formulations may contain one or more of the following agents: sweeteners, flavoring agents, coloring agents and preservatives. The sweetening and flavoring agents will increase the palatability of the preparation. Tablets containing extracts in admixture with non-toxic pharmaceutically acceptable excipients suitable for tablet manufacture are acceptable. Pharmaceutically acceptable means that the agent should be acceptable in the sense of being compatible with the other ingredients of the formulation (as well as non-injurious to the patient). Such excipients include inert diluents such as calcium carbonate, sodium carbonate, lactose, calcium phosphate or sodium phosphate; granulating and disintegrating agents, such as corn starch or alginic acid; binding agents such as starch, gelatin or acacia; and lubricating agents such as magnesium stearate, stearic acid or talc. Tablets can be uncoated or can be coated by known techniques to delay disintegration and absorption in the gastrointestinal tract and thereby provide a sustained action over a longer period of time. For example, a time delay material such as glyceryl monostearate or glyceryl distearate alone or with a wax may be employed.

Formulations for oral use can also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, such as peanut oil, liquid paraffin or olive oil. In some embodiments, aqueous suspensions can contain an extract of the invention in admixture with excipients suitable for the manufacture of aqueous suspensions. Such excipients include, but are not limited to, suspending agents, dispersing or wetting agents, one or more preservatives, one or more coloring agents, one or more flavoring agents and one or more sweetening agents such as sucrose or saccharin. Oil suspensions may be formulated by suspending the active ingredient in a vegetable oil, such as arachis oil, olive oil, sesame oil or coconut oil, or in a mineral oil such as liquid paraffin. The oil suspension may contain a thickening agent, such as beeswax, hard paraffin or cetyl alcohol. Sweetening agents, such as those set forth above, and flavoring agents may be added to provide a palatable oral preparation. These formulations may be preserved by an added antioxidant such as ascorbic acid. Dispersible powders and granules of the invention suitable for preparation of an aqueous suspension by the addition of water provide one or more extracts in admixture with a dispersing or wetting agent, a suspending agent, and one or more preservatives. Additional excipients, for example sweetening, flavoring and coloring agents, may also be present.

Syrups and elixirs may be formulated with sweetening agents, such as glycerol, sorbitol or sucrose. Such formulations may also contain a demulcent, a preservative, a flavoring or a coloring agent.

The extract preparations for parenteral administration may be in the form of a sterile injectable preparation, such as a sterile injectable aqueous or oleaginous suspension. This suspension may be formulated according to methods well known in the art using suitable dispersing or wetting agents and suspending agents. The sterile injectable preparation may also be a sterile injectable solution or suspension in a non-toxic parenterally-acceptable diluent or solvent, such as a solution in 1,3-butanediol. Suitable diluents include, for example, water, Ringer's solution and isotonic sodium chloride solution. In addition, sterile fixed oils may be employed conventionally as a solvent or suspending medium. For this purpose, any bland fixed oil may be employed including synthetic mono or diglycerides. In addition, fatty acids such as oleic acid may likewise be used in the preparation of injectable preparations.

The pharmaceutical formulations may also be in the form of oil-in-water emulsions. The oily phase may be a vegetable oil, such as olive oil or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions may also contain sweetening and flavoring agents.

The amount of extract that may be combined with the carrier material to produce a single dosage form will vary depending upon the host treated and the particular mode of administration.

While the present invention may be a pharmaceutical product, it can also be consumed as a nutriceutical, that is, a nutritional or health supplement designed for any specific clinical purpose. As such, it can be for general consumption as a nutritional or health supplement.

Variations within the Scope of the Invention

According to traditional Chinese medicine practice and the stipulations in the Pharmacopoeia of the People's Republic of China, dried plant materials are typically preferred for use. Thus, all weights referring to herbs in the above description are the dry weights of those herbs. While dried material is traditionally used and preferred, it must be recognized that drying of plant materials facilitates their storage, transportation and subsequent processing. Drying may not be a requirement to derive the benefits of these herbs. As such, the present invention may be practiced with the listed fresh plant materials as well. The use of fresh plant materials, sufficient to meet the requisite quantity and proportions of the extracts used, come under the scope of the present invention.

It is understood that several species within a plant genus may be given under a particular plant's entry in the Pharmacopoeia and these species with the same genus may be freely substituted by, or used in conjunction with, other members of the same genus as given in the Pharmacopoeia.

In addition, it is recognized that certain plant parts may contain the active components of interest in higher concentration and the present invention teaches the use of specific plant parts under the standardized nomenclature of the Pharmacopoeia. However these components may also be present in the other parts of the same plants. As such, the components of interest may also be extracted from other parts of the same plant under the scope of the present claims. A person skilled in the art will appreciate that it is possible, with plant cell and tissue culture techniques, to culture the cells and tissue of these herbs in vitro and to extract the active components of interest from these cells and tissue.

The extraction process entailed reducing the size of the herbal materials. Here, the reducing in size may be achieved by a number of ways including, but not limited to, cutting, chopping, mincing, pounding, pulverizing, milling and grinding. While one way may be taught, other ways and means of achieving a reduction in size of the materials may also be used.

Although the foregoing invention has been described in some detail by way of illustration and example for purposes of clarity of understanding, only the preferred embodiments were described. Certain changes and modifications may be made thereto without departing from the spirit and scope of that which is described and claimed. Utilizing the description above, a person skilled in the art of the preparation and use of Chinese herbal medicine can readily practice the methods of the present invention.

1. A formulation comprising the extracts of plant materials Sinomenium spp., Aconitum carmichaeli Debx., Paeonia lactiflora Pall., Paeonia suffruticosa Andr. and Curcuma longa L.

2. The formulation of claim 1 consisting essentially of the extracts of the plant materials Sinomenium spp., Aconitum carmichaeli Debx., Paeonia lactiflora Pall., Paeonia suffruticosa Andr. and Curcuma longa L.

3. The formulation of claim 1 consisting of the extracts of the plant materials Sinomenium spp., Aconitum carmichaeli Debx., Paeonia lactiflora Pall., Paeonia suffruticosa Andr. and Curcuma longa L.

4. The formulation of claim 1, wherein the Sinomenium spp. is selected from the group consisting of Sinomenii acutum (Thunb.) Rehd. et Wils. and Sinomenii acutum (Thunb.) Rehd. et Wils. var. cinereum Rehd. et Wils.

5. The formulation of claim 1, wherein the plant materials are Caulis Sinomenii, Radix Aconitii Lateralis Preparata, Radix Paeoniae Alba, Cortex Moutan and Rhizoma Curcumae Longae.

6. The formulation of claim 5, wherein the plant materials are in the proportion by weight of about: Caulis Sinomenii 2-10 parts, Radix Aconitii Lateralis Preparata 1-6 parts, Radix Paeoniae Alba 3-15 parts, Cortex Moutan 1-8 parts and Rhizoma Curcumae Longae 1-8 parts.

7. The formulation of claim 5, wherein the plant materials are in the proportion by weight of about: Caulis Sinomenii 5 parts, Radix Aconiti Lateralis Preparata 3 parts, Radix Paeoniae Alba 6 parts, Cortex Moutan 3 parts, and Rizhoma Curcumae Longae 3 parts.

8. The formulation of claim 5, wherein the plant materials are in the proportion by weight of about: Caulis Sinomenii 4 parts, Radix Aconiti Lateralis Preparata 3 parts, Radix Paeoniae Alba 5 parts, Cortex Moutan 3 parts, and Rizhoma Curcumae Longae 2 parts.

9. The formulation of claim 6 further comprising a pharmaceutically acceptable carrier, diluent or additive.

10. The formulation of claim 6, wherein the formulation is in a form suitable for oral administration.

11. The formulation of claim 10, wherein the form is selected from the group consisting of capsule, powder, tablet, liquid and caplet.

12. A formulation effective for antiarthritis, antiinflammation or antinotiception comprising therapeutically effective amounts of sinomenine, paeoniflorin, peonol and curcumin in the following proportions by weight: sinomenine 1-5 parts, paeoniflorin 5-28 parts, peonol 1-10 parts and curcumin 1-8 parts.

13. The formulation according to claim 12, further comprising therapeutically effective amounts of demethoxycurcumin and bisdemethoxycurcumin.

14. A method for preparing the formulation of claim 5, comprising:
a. reducing the size of Caulis Sinomenii, Radix Aconitii Lateralis Preparata, Radix Paeoniae Alba, Cortex Moutan and Rhizoma Curcumae Longae;
b. extracting the size-reduced plant materials obtained from step a more than once with suitable liquids to obtain extracts;
c. concentrating the extracts obtained from step b; and
d. combining the above concentrated extracts.

15. The method of claim 14, wherein step b comprises:
(i) extracting Caulis Sinomenii, Radix Aconitii Lateralis Preparata, and Radix Paeoniae Alba together with an alcohol to produce an Extract 1;
(ii) extracting Cortex Moutan with supercritical carbon dioxide to produce an Extract 2; and
(iii) extracting Rhizoma Curcumae Longae with supercritical carbon dioxide to produce an Extract 3.

16. The method of claim 15, wherein the Cortex Moutan extracting step further comprises extracting the Cortex Moutan residue with an alcohol to produce another extract.

17. The method of claim 15, wherein the Rhizoma Curcumae Longae extracting step further comprises extracting the Rhizoma Curcumae Longae residue with an alcohol to produce another extract.

18. The method of claim 15 wherein the plant materials are in proportion by weight of about:
Caulis Sinomenii 2-10 parts, Radix Aconiti Lateralis Preparata 1-6 parts and Radix Paeoniae Alba 3-15 parts, Cortex Moutan 1-8 parts and Rizhoma Curcumae Longae 1-8 parts.

19. (canceled)

20. The method of claim 14 further comprising subjecting at least one of the extracts from the step b to a purification process, wherein the purification process is passage through a polymeric adsorption resin.

21. The method of claim 14 wherein the plant materials are in proportion by weight of about: Caulis Sinomenii 4 parts, Radix Aconiti Lateralis Preparata 3 parts, Radix Paeoniae Alba 5 parts, Cortex Moutan 3 parts, and Rizhoma Curcumae Longae 2 parts; and
Step b further comprises:
(i) extracting Caulis Sinomenii with water to obtain an aqueous Caulis Sinomenii extract as an Extract 1.1; and
extracting Radix Aconiti Lateralis Preparata and Radix Paeoniae Alba with an alcohol to obtain an alcoholic extract of Radix as an Extract 1.2;
(ii) extracting Cortex Moutan with supercritical carbon dioxide to obtain a supercritical fluid extract of Cortex Moutan an Extract 2.1; and
extracting Cortex Moutan residue from the above supercritical fluid extraction step with an alcohol to obtain alcoholic Cortex Moutan extract as an Extract 2.2; and
(iii) extracting Rizhoma Curcumae Longae with supercritical carbon dioxide to obtain a supercritical fluid extract of Rizhoma Curcumae Longae as an Extract 3.1; and
extracting Rizhoma Curcumae Longae residue from the above supercritical fluid extraction step with an alcohol to obtain an alcoholic extract of Rizhoma Curcumae Longae as an Extract 3.2;
wherein step c further comprises:
concentrating each of the above Extracts from step b to obtain Concentrated Extracts; and
wherein step d further comprises:
combining the above Concentrated Extracts with suitable excipients to obtain the formulation.

22. The method of claim 21 wherein the aqueous and alcoholic extractions of step b are performed more than once and the extracts from the multiple extractions are combined and filtered to obtain Filtered Extracts; and
step c further comprises:
(1) concentrating each of the above Filtered Extracts from step b to a suitable relative density;
(2) drying the concentrated Filtered Extract from the Radix Aconiti Lateralis Preparata and Radix Paeoniae Alba to obtain a Radix Powder Extract;
(3) adding egg white to the concentrated Filtered Extracts from Caulis Sinomenii, Cortex Moutan, respectively, to form an egg white mixture; boiling and centrifugating the egg white mixture; removing the supernatant and concentrating it to a suitable relative density to obtain a concentrated filtrate; adding alcohol to the concentrated filtrate to obtain an alcoholic mixture with a liquid fraction and a solid fraction; concentrating the liquid fraction; and adding β-cyclodexrin to the concentrated liquid fraction; drying the above concentrated liquid fraction to form a Caulis Sinomenii Powder Extract and a Cortex Moutan Powder Extract; and
(4) adding an excipient to the concentrated Filtered Extract from the Rizhoma Curcumae Longae and drying said Rizhoma Extract to form a Rizhoma Powder Extract; further wherein
step d further comprises:
(1) combining the above Powder Extracts with suitable excipients to form a powder mixture; and
(2) granulating the Powder Mixture to form a granule mixture;
filling capsules with the granule mixture and suitable excipients.

23. The formulation of claim 6, wherein the formulation is made by a process comprising:
a. reducing the size of Caulis Sinomenii, Radix Aconitii Lateralis Preparata, Radix Paeoniae Alba, Cortex Moutan and Rhizoma Curcumae Longae;
b. extracting the size-reduced plant materials obtained from step a more than once with suitable liquids to obtain extracts;
c. concentrating the extracts obtained from the step b; and
d. combining the above concentrated extracts.

24. The formulation of claim 23, wherein the extracting step comprises:
(i) extracting Caulis Sinomenii, Radix Aconitii Lateralis Preparata and Radix Paeoniae Alba together with an alcohol to produce an Extract 1;
(ii) extracting Cortex Moutan with supercritical carbon dioxide to produce an Extract 2; and
(iii) extracting Rhizoma Curcumae Longae with supercritical carbon dioxide to produce an Extract 3.

25. The formulation of claim 24, wherein the Cortex Moutan extracting step (ii) further comprises extracting the Cortex Moutan residue with an alcohol to produce another extract.

26. (canceled)

27. The formulation of claim 25, wherein the process further comprises subjecting at least one of the extracts from the step b to a purification process, wherein the purification process comprises a passage through a polymeric adsorption resin.

28. A method for treating arthritis or arthritis-associated inflammation and pain in a mammal comprising administering a therapeutically effective amount of the formulation of claim 6.

29. The method of claim 28 wherein the arthritis is rheumatoid arthritis.

30. The method of claim 28 wherein the arthritis is ankylosing spondylitis.

31. The method of claim 28 wherein the mammal is a human being.

32. A method for treating arthritis and similar conditions comprising administering a therapeutically effective amount of the formulation of claim 5.

33. A nutriceutical comprising the formulation of claim 1.

34. A method for ascertaining the quality of the formulation of claim 1 comprising:
(a) providing an extract of the formulation;
(b) subjecting the extract to at least one separation technique under certain parameters;
(c) subjecting at least one known marker to the same separation technique under the same parameters as the above;
(d) comparing the at least one known marker with a corresponding marker in the extract to ascertain the quality of the formulation.

35. The method of claim 34 wherein said separation technique is selected from the group consisting of thin layer chromatography, high performance liquid chromatography and a combination thereof.

36. The method according to claim 34 wherein the comparing step comprises qualitative comparisons and quantitative comparisons.

37. The formulation of claim 7 wherein the formulations consist essentially of the extracts of the plant materials in the proportion by weight of about: 5 parts of Caulis Sinomeni extract, 3 parts of Radix Aconitii Lateralis Preparata extract, 6 parts of Radix Paeoniae Alba extract, 3 parts of Cortex Moutan extract, and 3 parts of Rhizoma Curcumae Longae extract.

38. The method of claim 18, wherein the plant materials are in proportion by weight of about: Caulis Sinomenii 5 parts, Radix Aconiti Lateralis Preparata 3 parts and Radix Paeoniae Alba 6 parts, Cortex Moutan 3 parts and Rizhoma Curcumae Longae 3 parts.

39. The method of claim 18 wherein
step (i) further comprises:
(1) extracting Caulis Sinomenii, Radix Aconiti Lateralis Preparata and Radix Paeoniae Alba with ethanol more than once to obtain ethanolic Extracts 1; and
(2) combining and filtering Ethanolic Extracts 1 to obtain filtered Extracts;
step (ii) further comprises:
(1) extracting Cortex Moutan with supercritical carbon dioxide to produce an Extract 2;
(2) extracting the Cortex Moutan residue from the above supercritical extraction with ethanol more than once to obtain ethanolic Cortex Moutan extracts; and
(3) combining and filtering said Cortex Moutan extracts to obtain filtered Cortex Moutan extracts;
step (iii) further comprises:
(1) extracting Rhizoma Curcumae Longae with supercritical carbon dioxide to produce an Extract 3;
(2) extracting the Rizhoma Curcumae Longae residue from the above (1) with ethanol more than once to obtain ethanolic Rizhoma Curcumae Longae extracts; and
(3) combining and filtering said ethanolic Rizhoma Curcumae Longae extracts to obtain filtered Rizhoma Curcumae Longae extracts;
wherein step c further comprises concentrating the filtered Extracts obtained from the step (i) to (iii) to obtain Concentrated Extracts 1a, 2a and 3a, respectively; and
wherein step d further comprises combining the above Concentrated Extracts 1a, 2a, 3a with the Extract 2 and Extract 3 to obtain the formulation.
